(19) United States
(12) Patent Application Publication
(10) Pub. No.: US 2006/0173048 A1
(43) Pub. Date: Aug. 3, 2006

(54) Imidazole derivatives

(76) Inventors: Silvia Gatti McArthur, Basel (CH); Erwin Goetschi, Reinach (CH); Juergen Wichmann, Steinen (DE)

Correspondence Address: HOFFMANN-LA ROCHE INC.;PATENT LAW DEPARTMENT, 340 KINGSLAND STREET, NUTLEY, NJ, 07110, US

(21) Appl. No.: 11/341,704

(22) Filed: Jan. 27, 2006

(30) Foreign Application Priority Data
Feb. 3, 2005 (EP) 05100751.6

(51) Int. Cl. A61K 31/4439; C07D 403/04

(52) U.S. Cl. 514/341; 546/272.7

(57) ABSTRACT

The invention relates to compounds of formula I: 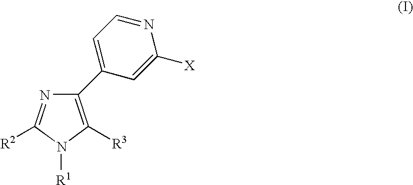
wherein R¹, R², R³ and X are as defined in the specification, methods of preparation thereof and uses thereof, which compounds are useful for preventing and treating mGluR 2 receptor mediated disorders.

### PRIORITY TO RELATED APPLICATIONS

This application claims the benefit of European Application No. 05100751.6 filed Feb. 3, 2005, which is hereby incorporated by reference in its entirety.

### BACKGROUND OF THE INVENTION

In the central nervous system (CNS) the transmission of stimuli takes place by the interaction of a neurotransmitter, which is sent out by a neuron, with a neuroreceptor.

L-glutamic acid, the most commonly occurring neurotransmitter in the CNS, plays a critical role in a large number of physiological processes. The glutamate-dependent stimulus receptors are divided into two main groups. The first main group forms ligand-controlled ion channels. The metabotropic glutamate receptors (mGluR) form the second main group and, furthermore, belong to the family of G-protein-coupled receptors.

At present, eight different members of these mGluR are known and of these some even have sub-types. On the basis of structural parameters, the different influences on the synthesis of secondary metabolites and the different affinity to low-molecular weight chemical compounds, these eight receptors can be subdivided into three sub-groups: mGluR1 and mGluR5 belong to group I, mGluR2 and mGluR3 belong to group II and mGluR4, mGluR6, mGluR7 and mGluR8 belong to group III.

Ligands of metabotropic glutamate receptors belonging to the group II can be used for the treatment or prevention of acute and/or chronic neurological disorders such as psychosis, schizophrenia, Alzheimer's disease, cognitive disorders and memory deficits. Other treatable indications in this connection are restricted brain function caused by bypass operations or transplants, poor blood supply to the brain, spinal cord injuries, head injuries, hypoxia caused by pregnancy, cardiac arrest and hypoglycaemia. Further treatable indications are chronic and acute pain, Huntington's chorea, amyotrophic lateral sclerosis (ALS), dementia caused by AIDS, eye injuries, retinopathy, idiopathic parkinsonism or parkinsonism caused by medicaments as well as conditions which lead to glutamate-deficiency functions, such as e.g. muscle spasms, convulsions, migraine, urinary incontinence, nicotine addiction, opiate addiction, anxiety, vomiting, dyskinesia, depressions and glioma, since mGluR2 antagonists have been found to reduce cell proliferation in human glioma cells (J. Neurochem. March 2003, 84(6): 1288-95).

### SUMMARY OF THE INVENTION

The present invention provides compounds of general formula I 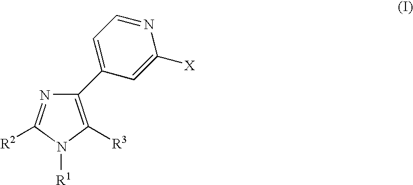
wherein
R¹ is a CHR^{a}R^{b} group in which:

either R^{a} is H and R^{b} is C₁₋₅-alkyl, C₂₋₅-alkenyl or C₂₋₅-alkynyl, each of which is optionally substituted by 1 to 5 substituents selected from the group consisting of F, Cl, Br, OH and OMe;

or R^{a} and R^{b} together with the carbon atom to which they are attached form a C₃₋₆-cycloalkyl ring; wherein R^{a} and R^{b} are each optionally substituted by 1 to5 substituents selected from the group consisting of F, Cl, Br, OH and OMe;
R² is tert.-butyl;

or is —CR^{c}R^{d}R^{e}; wherein R^{c} and R^{d} are each independently branched or linear C₂₋₅-alkyl, C₂₋₅-alkenyl, C₃₋₆-cycloalkyl, phenyl, or benzyl, each of which is optionally substituted by 1 to 4 substituents selected from the group consisting of F, Cl, Br, OH, trifluoromethyl, C₁₋₄-alkyl and C₁₋₄-alkoxy; and R^{e} is H, OH, or a group OCH₂R^{f} wherein R^{f} is C₁₋₆-alkyl optionally substituted by OH;

or is aryl or heteroaryl, each of which is optionally substituted by 1 to 4 substituents selected from the group consisting of C₁₋₄-alkyl, trifluoromethyl, F, Cl, Br, OH, and C₁₋₄-alkoxy;
R³ is phenyl optionally substituted by 1 to 3 substituents selected from the group consisting of F, Cl, Br, trifluoromethyl and C₁₋₄-alkyl;
X is H, Cl, F or methoxy;
   and pharmaceutically acceptable salts thereof.

The invention includes all racemic mixtures, all their corresponding enantiomers and/or optical isomers. The present invention also provides pharmaceutical compositions containing compounds of the invention and a pharmaceutically acceptable carrier. The invention also provides methods for manufacturing compounds of the invention and compositions containing them.

Compounds of formula I are metabotropic glutamate receptor antagonists. The invention also relates to use of compounds of formula I, and pharmaceutically acceptable salts thereof for the treatment of mGluR 2 receptor mediated disorders.

### DETAILED DESCRIPTION OF THE INVENTION

The following definitions of the general terms used in the present description apply irrespective of whether the terms in question appear alone or in combination. It must be noted that, as used in the specification and the appended claims, the singular forms “a”, “an,” and “the” include plural forms unless the context clearly dictates otherwise.

Unless specified otherwise, the term “alkyl” used in the present description denotes straight-chain or branched saturated hydrocarbon residues with 1-8 carbon atoms, preferably with 1-6 carbon atoms. Examples of alkyl groups are methyl, ethyl, n-propyl, i-propyl, tert.-butyl, 3-pentyl, 4-heptyl, 5-heptyl, as well as those specifically exemplified in the instant patent application.

The term “C₁₋₄-alkoxy” denotes a group wherein the alkyl group is as defined above and is connected via an oxygen atom. Preferred C₁₋₄-alkoxy groups are methoxy or ethoxy.

The term “alkenyl” used in the present description denotes straight-chain or branched unsaturated hydrocarbon residues with 2-6, preferably 2-4 carbon atoms, such as ethenyl, 2-propenyl, isobutene-1-yl, and those specifically exemplified in the instant patent application.

The term “alkynyl” used in the present description denotes straight-chain or branched unsaturated hydrocarbon residues with 2-6, preferably 2-4 carbon atoms, such as ethynyl, n-propynyl, and those specifically exemplified in the instant patent application.

The term “aryl” used in the present description denotes an aromatic ring having 6 to 12 ring carbon atoms. The most preferred aryl is phenyl. Aryl can be substituted by one or more substituents including halogen, C₁₋₄-alkyl, trifluoromethyl, trifluoroethyl and C₁₋₄-alkoxy as well as the substituents specifically exemplified in the instant patent application.

The term “halogen” embraces fluorine, chlorine and bromine.

The term “C₃₋₆-cycloalkyl” denotes a carbon ring having 3 to 6 carbon atoms as ring members and includes but is not limited to cyclopropyl, cyclobutyl, cyclopentyl and cyclohexyl.

The term “heteroaryl” embraces a 5 or 6 membered aromatic heterocyclic ring containing 1-3 heteroatoms (N, O, S) preferrably annelated by a phenyl ring. Examples for such heteroaryl groups are 3-pyridyl, 2-indolyl, 4-quinolinyl and those specifically exemplified in the instant patent application.

The expression “R¹ is a CHR^{a}R^{b} group in which R^{a} and R^{b} together with the carbon atom to which they are attached form a C₃₋₆-cycloalkyl ring; wherein R^{a} and R^{b} are each optionally substituted by 1 to 5 substituents selected from the group consisting of F, Cl, Br, OH and OMe” means a 3-6 membered cycloalkyl ring of the following formula: 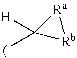
wherein the hereinabove recited optional substituents are attached to R^{a} and/or R^{b}.

“Pharmaceutically acceptable,” such as pharmaceutically acceptable carrier, excipient, etc., means pharmacologically acceptable and substantially non-toxic to the subject to which the particular compound is administered.

The term “pharmaceutically acceptable acid addition salts” embraces salts with inorganic and organic acids, such as hydrochloric acid, nitric acid, sulfuric acid, phosphoric acid, citric acid, formic acid, fumaric acid, maleic acid, acetic acid, succinic acid, tartaric acid, methane-sulfonic acid, p-toluenesulfonic acid and the like.

“Therapeutically effective amount” means an amount that is effective to prevent, alleviate or ameliorate symptoms of disease or prolong the survival of the subject being treated.

The present invention provides compounds of general formula I 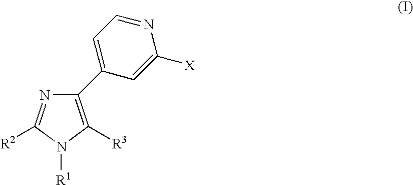
wherein
R¹ is a CHR^{a}R^{b} group in which:

either R^{a} is H and R^{b} is C₁₋₅-alkyl, C₂₋₅-alkenyl or C₂₋₅-alkynyl, each of which is optionally substituted by 1 to5 substituents selected from the group consisting of F, Cl, Br, OH and OMe;

or R^{a} and R^{b} together with the carbon atom to which they are attached form a C₃₋₆-cycloalkyl ring; wherein R^{a} and R^{b} are each optionally substituted by 1 to 5 substituents selected from the group consisting of F, Cl, Br, OH and OMe;
R² is tert.-butyl;

or is —CR^{c}R^{d}R^{e}; wherein R^{c} and R^{d} are each independently branched or linear C₂₋₅-alkyl, C₂₋₅-alkenyl, C₃₋₆-cycloalkyl, phenyl, or benzyl, each of which is optionally substituted by 1 to 4 substituents selected from the group consisting of F, Cl, Br, OH, trifluoromethyl, C₁₋₄-alkyl and C₁₋₄-alkoxy; and R^{e} is H, OH, or a group OCH₂R^{f} wherein R^{f} is C₁₋₆-alkyl optionally substituted by OH;

or is aryl or heteroaryl, each of which is optionally substituted by 1 to 4 substituents selected from the group consisting of C₁₋₄-alkyl, trifluoromethyl, F, Cl, Br, OH, and C₁₋₄-alkoxy;
R³ is phenyl optionally substituted by 1 to 3 substituents selected from the group consisting of F, Cl, Br, trifluoromethyl and C₁₋₄-alkyl;
X is H, Cl, F or methoxy;
   and pharmaceutically acceptable salts thereof.

In certain embodiments of the invention, the compounds of the invention are those compounds wherein:
R¹ is a CHR^{a}R^{b} group in which R^{a} is H and R^{b} is C₁₋₅-alkyl, C₂₋₅-alkenyl or C₂₋₅-alkynyl, each of which is optionally substituted by 1 to 5 substituents selected from the group consisting of F, Cl, Br, OH and OMe;

or R^{a} and R^{b} together with the carbon atom to which they are attached form a C₃₋₆-cycloalkyl ring; wherein R^{a} and R^{b} are each optionally substituted by 1 to 5 substituents selected from the group consisting of F, Cl, Br, OH and OMe;
R² is tert.-butyl;

—CR^{c}R^{d}R^{e}; wherein R^{c} and R^{d} are each independently branched or linear C₂₋₅-alkyl, C₂₋₅-alkenyl, C₃₋₆-cycloalkyl, phenyl, or benzyl, each of which is optionally substituted by 1 to 4 substituents selected from the group consisting of F, Cl, Br, trifluoromethyl, C₁₋₄-alkyl and C₁₋₄-alkoxy; and R^{e} is H, OH, or a group OCH₂R^{f} wherein R^{f} is C₁₋₆-alkyl optionally substituted by OH;

aryl or heteroaryl, each of which is optionally substituted by 1 to 4 substituents selected from the group consisting of C₁₋₄-alkyl, trifluoromethyl, F, Cl, Br, OH, and C₁₋₄-alkoxy;
R³ is phenyl optionally substituted by 1 to 3 substituents selected from the group consisting of F, Cl, Br, trifluoromethyl and C₁₋₄-alkyl;
X is H, Cl, F or methoxy;
   and pharmaceutically acceptable salts thereof;

In certain embodiments the compounds of the invention are those compounds of formula I wherein R² is tert.-butyl, for example the following compound:
4-[2-tert-Butyl-5-(4-chloro-phenyl)-1-ethyl-1H-imidazol-4-yl]-pyridine.

In certain embodiments the compounds of the invention are those compounds of formula I wherein R² is —CR^{c}R^{d}R^{e}, wherein R^{c} and R^{d} are each independently branched or linear C₂₋₅-alkyl, C₂₋₅-alkenyl, C₃₋₆-cycloalkyl, phenyl, or benzyl, each of which is optionally substituted by 1 to 4 substituents selected from the group consisting of F, Cl, Br, trifluoromethyl, C₁₋₄-alkyl and C₁₋₄-alkoxy; and R^{e} is H, OH, or a group OCH₂R^{f} wherein R^{f} is C₁₋₆-alkyl optionally substituted by OH; for example the following compounds:
4-[5-(4-Chloro-phenyl)-1-ethyl-2-(1-ethyl-propyl)-1H-imidazol-4-yl]-pyridine;
4-[5-(4-Chloro-phenyl)-2-(1-ethyl-propyl)-1-methyl-1H-imidazol-4-yl]-pyridine;
4-[5-(4-Chloro-phenyl)-1-ethyl-2-(1-propyl-butyl)-1H-imidazol-4-yl]-pyridine;
4-[5-(4-Chloro-phenyl)-1-ethyl-2-(1-isobutyl-3-methyl-butyl)-1H-imidazol-4-yl]-pyridine;
4-[2-Benzhydryl-5-(4-chloro-phenyl)-1-ethyl-1H-imidazol-4-yl]-pyridine;
3-[5-(4-Chloro-phenyl)-1-ethyl-4-pyridin-4-yl-1H-imidazol-2-yl]-pentan-3-ol;
3-[5-(4-Chloro-phenyl)-1-ethyl-4-pyridin-4-yl-1H-imidazol-2-yl]-2,4-dimethyl-pentan-3-ol;
4-[5-(4-Chloro-phenyl)-1-ethyl-4-pyridin-4-yl-1H-imidazol-2-yl]-hepta-1,6-dien-4-ol;
[5-(4-Chloro-phenyl)-1-ethyl-4-pyridin-4-yl-1H-imidazol-2-yl]-dicyclopropyl-methanol;
[5-(4-Chloro-phenyl)-1-ethyl-4-pyridin-4-yl-1H-imidazol-2-yl]-diphenyl-methanol;
[5-(4-Chloro-phenyl)-1-ethyl-4-pyridin-4-yl-1H-imidazol-2-yl]-bis-(3-trifluoromethyl-phenyl)-methanol;
[5-(4-Chloro-phenyl)-1-ethyl-4-pyridin-4-yl-1H-imidazol-2-yl]-bis-(3-fluoro-phenyl)-methanol;
[5-(4-Chloro-phenyl)-1-ethyl-4-pyridin-4-yl-1H-imidazol-2-yl]-dicyclohexyl-methanol;
2-[5-(4-Chloro-phenyl)-1-ethyl-4-pyridin-4-yl-1H-imidazol-2-yl]-1,3-diphenyl-propan-2-ol and
4-[2-[Bis-(3-fluoro-phenyl)-methyl]-5-(4-chloro-phenyl)-1-ethyl-1H-imidazol-4-yl]-pyridine.

In certain embodiments the compounds of the invention are those of formula I wherein, R² is —CR^{c}R^{d}R^{e}, wherein R^{c} and R^{d} are each independently phenyl or benzyl, each of which is optionally substituted by 1 to 4 substituents selected from the group consisting of F, Cl, Br, trifluoromethyl, C₁₋₄-alkyl and C₁₋₄-alkoxy, and R^{e} is H, OH, or a group OCH₂R^{f} wherein R^{f} is C₁₋₆-alkyl optionally substituted by OH.

In certain embodiments the compounds of the invention are those compounds of formula I wherein R² is optionally substituted aryl or heteroaryl, which when substituted is substituted by 1 to 4 substituents selected from the group consisting of C₁₋₄-alkyl, trifluoromethyl, F, Cl, Br and C₁₋₄-alkoxy; for example the following compounds:
4-[2-(2,6-Dichloro-phenyl)-1-methyl-5-(4-methyl-phenyl)-1H-imidazol-4-yl]-pyridine;
4-[2-(2,6-Dichloro-phenyl)-1-propyl-5-(4-methyl-phenyl)-1H-imidazol-4-yl]-pyridine;
4-[1-Methyl-5-(4-methyl-phenyl)-2-(2,4,6-trimethyl-phenyl)-1H-imidazol-4-yl]-pyridine;
4-[5-(4-Chloro-phenyl)-2-(2,6-dichloro-phenyl)-1-methyl-1H-imidazol-4-yl]-pyridine;
4-[1-Ethyl-5-(4-methyl-phenyl)-2-(2,4,6-trimethyl-phenyl)-1H-imidazol-4-yl]-pyridine;
4-[5-(4-Chloro-phenyl)-2-(2,6-dichloro-phenyl)-1-ethyl-1H-imidazol-4-yl]-pyridine;
4-[1-Ethyl-5-(4-trifluoromethyl-phenyl)-2-(2,4,6-trimethyl-phenyl)-1H-imidazol-4-yl]-pyridine;
4-[5-(4-tert-Butyl-phenyl)-1-ethyl-2-(2,4,6-trimethyl-phenyl)-1H-imidazol-4-yl]-pyridine;
4-[5-(4-Chloro-phenyl)-1-ethyl-2-(2-methyl-phenyl)-1H-imidazol-4-yl]-pyridine;
4-[5-(4-Chloro-phenyl)-1-ethyl-2-phenyl-1H-imidazol-4-yl]-pyridine;
4-[1-Ethyl-5-(4-fluoro-phenyl)-2-(2,4,6-trimethyl-phenyl)-1H-imidazol-4-yl]-pyridine;
4-[2-(2,6-Dichloro-phenyl)-1-ethyl-5-(4-methyl-phenyl)-1H-imidazol-4-yl]-pyridine;
4-[5-(4-Chloro-phenyl)-1-ethyl-2-(2,4,6-trimethyl-phenyl)-1H-imidazol-4-yl]-pyridine;
4-[1-Ethyl-5-(4-trifluoromethyl-phenyl)-2-(2,4,6-trimethyl-phenyl)-1H-imidazol-4-yl]-pyridine;
4-[5-(4-Chloro-phenyl)-2-(2,6-dimethyl-phenyl)-1-ethyl-1H-imidazol-4-yl]-pyridine;
4-[5-(4-Chloro-phenyl)-2-(2-chloro-phenyl)-1-ethyl-1H-imidazol-4-yl]-pyridine;
4-[2-(2-Bromo-phenyl)-5-(4-chloro-phenyl)-1-ethyl-1H-imidazol-4-yl]-pyridine;
4-[2-(2-Chloro-6-methyl-phenyl)-5-(4-chloro-phenyl)-1-ethyl-1H-imidazol-4-yl]-pyridine;
4-[5-(4-Chloro-phenyl)-1-ethyl-2-(2,4,6-trimethoxy-phenyl)-1H-imidazol-4-yl]-pyridine;
4-[1-Allyl-2-(2,6-dichloro-phenyl)-5-(4-methyl-phenyl)-1H-imidazol-4-yl]-pyridine;
4-[1-Allyl-5-(4-trifluoromethyl-phenyl)-2-(2,4,6-trimethyl-phenyl)-1H-imidazol-4-yl]-pyridine;
4-[1-Allyl-5-(4-chloro-phenyl)-2-(2,4,6-trimethyl-phenyl)-1H-imidazol-4-yl]-pyridine;
4-[1-Allyl-5-(4-chloro-phenyl)-2-(2,6-dichloro-phenyl)-1H-imidazol-4-yl]-pyridine
4-[1-Allyl-5-(4-methyl-phenyl)-2-(2,4,6-trimethyl-phenyl)-1H-imidazol-4-yl]-pyridine;
4-[5-(4-Chloro-phenyl)-1-cyclopentyl-2-(2,6-dichloro-phenyl)-1H-imidazol-4-yl]-pyridine;
4-[5-(4-Chloro-phenyl)-2-(2,6-dichloro-phenyl)-1-(2,2,2-trifluoro-ethyl)-1H-imidazol-4-yl]-pyridine;
2-[5-(4-Chloro-phenyl)-4-pyridin-4-yl-2-(2,4,6-trimethyl-phenyl)-imidazol-1-yl]-ethanol;
4-[5-(4-Chloro-phenyl)-1-cyclopropyl-2-(2,6-dichloro-phenyl)-1H-imidazol-4-yl]-pyridine;
4-[5-(4-Chloro-phenyl)-2-(2,6-dichloro-phenyl)-1-prop-2-ynyl-1H-imidazol-4-yl]-pyridine;
3-[5-(4-Chloro-phenyl)-2-(2,6-dichloro-phenyl)-4-pyridin-4-yl-imidazol-1-yl]-propan-1-ol;
4-[2-(2,4-Bis-trifluoromethyl-phenyl)-5-(4-chloro-phenyl)-1-ethyl-1H-imidazol-4-yl]-pyridine;
3-[5-(4-Chloro-phenyl)-1-ethyl-4-pyridin-4-yl-1H-imidazol-2-yl]-pyridine;
4-[5-(4-Chloro-phenyl)-1-ethyl-4-pyridin-4-yl-1H-imidazol-2-yl]-quinoline;
2-[5-(4-Chloro-phenyl)-1-ethyl-4-pyridin-4-yl-1H-imidazol-2-yl]-1H-indole;
4-[5-(4-Chloro-phenyl)-2-(2,6-dichloro-phenyl)-1-propyl-1H-imidazol-4-yl]-pyridine;
4-[2-(2,6-Dichloro-phenyl)-1-propyl-5-(4-methyl-phenyl)-1H-imidazol-4-yl]-pyridine;
4-[1-Propyl-5-p-tolyl-2-(2,4,6-trimethyl-phenyl)-1H-imidazol-4-yl]-pyridine;
2-Chloro-4-[5-(4-chloro-phenyl)-2-(2,6-dichloro-phenyl)-1-ethyl-1H-imidazol-4-yl]-pyridine and
4-[5-(4-Chloro-phenyl)-2-(2,6-dichloro-phenyl)-1-ethyl-1H-imidazol-4-yl]-2-methoxy-pyridine.

In certain embodiments, the compounds of the invention are those in which R¹ is a CHR^{a}R^{b} group in which R^{a} is H and R^{b} is C₁₋₅-alkyl, C₂₋₅-alkenyl or C₂₋₅-alkynyl, each of which is optionally substituted by 1 to 5 substituents selected from the group consisting of F, Cl, Br, OH and OMe. In particular, the invention provides compounds within this group wherein R^{b} is C₁₋₅-alkyl.

In certain embodiments, the compounds of the invention are those in which R¹ is a CHR^{a}R^{b} group in which R^{a} and R^{b} together with the carbon atom to which they are attached form a C₃₋₆-cycloalkyl ring; wherein R^{a} and R^{b} are each optionally substituted by 1 to 5 substituents selected from the group consisting of F, Cl, Br, OH and OMe.

In a certain embodiment, the invention relates to a method for the preparation of the compounds of formula I according to the invention, said method comprising the steps of:

a) reacting a compound of formula V: 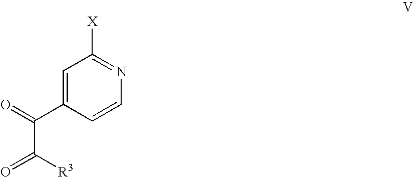
with a compound of formula VI:
R²—CHO (VI)
in order to obtain a compound of formula VII:

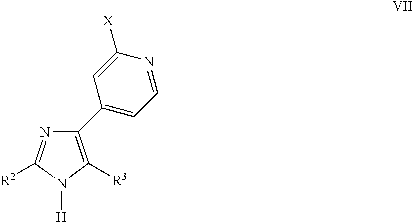

b) reacting the compound of formula VII with a compound of formula VIII:
R¹-L (VIII)
in order to obtain a compound of formula I, wherein R¹ to R³ and X are as defined herein above.

More details for this embodiment can be found in scheme 1 hereafter and in the description of procedure A.

In another embodiment, the invention relates to a method for the preparation of the compounds of formula I according to the invention, said method comprising the steps of:

a) reacting a compound of formula V: 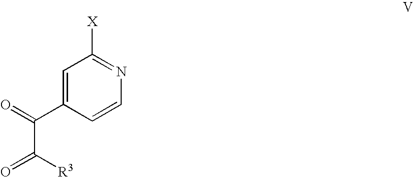
with compounds of formulae VI and IX:
R²—CHO (VI),
R¹—NH₂ (IX)
in order to obtain the compound of formula I, wherein R¹ to R³ and X are as defined herein above.

More details for this embodiment can be found in scheme 1 hereafter and in the description of procedure B.

In still another embodiment, the invention relates to a method for the preparation of the compounds of formula I according to the invention, said method comprising the steps of:

a) reacting a compound of formula X: 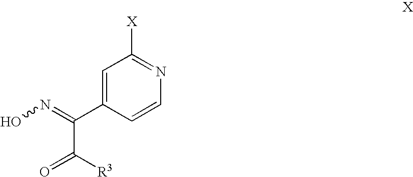
with compounds of formulae VI and IX:
R²—CHO (VI),
R¹—NH₂ (IX)
in order to obtain the compound of formula XI:

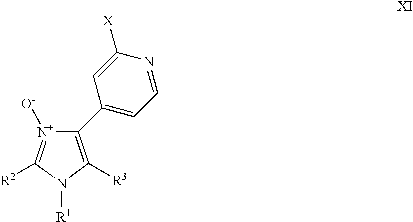

b) and reducing the compound of formula XI in order to obtain a compound of formula I, wherein R¹ to R³ and X are as defined herein above.

More details for this embodiment can be found in scheme 1 hereafter and in the description of procedure C.

As mentioned hereinabove, the compounds of general formula I can be carried out in accordance with the following general procedures A-C which are outlined below in scheme 1.

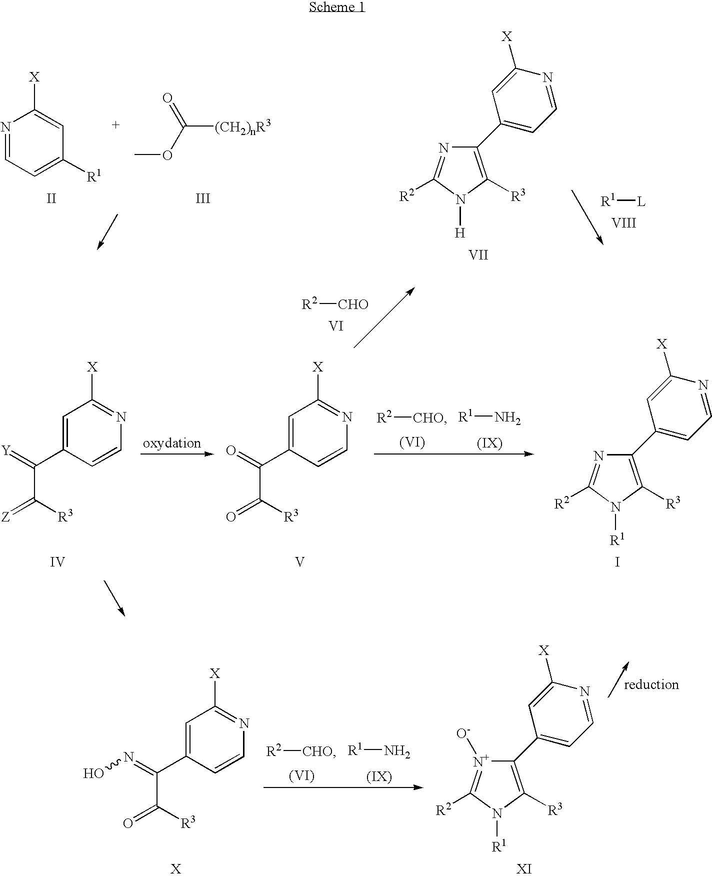

Procedure A: Compounds of formula I, in which R¹, R², R³ and X are as defined hereinabove can be prepared by the alkylation of an imidazole of the formula VII, in which R², R³ and X are as defined hereinabove, with an alkylating agents R¹-L (VIII), in which R¹ is as defined hereinabove and L is a leaving group, such as bromide, iodide or mesylate. The alkylation reaction is preferably carried out in an inert solvent, such as N,N-dimethyl formamide or DMSO, in the presence of a base, such as sodium hydride, at 0-100° C. Procedure A leads in general to a mixture of two products which comprises of a compound of formula I and a regioisomeric compound resulting from the non-specific alkylation reaction of the imidazole entity. In general, compounds of formula I are the major product in this reaction and can be isolated in pure form by chromatography or crystallization of the crude reaction product.

Imidazoles of formula VII can be prepared according to generally known methods, e.g. by heating a dione of formula V and an aldehyde of formula VI, in which R², R³ and X are as defined hereinabove, together with ammonium acetate in acetic acid to 100-110° C. for 1 to 5 h. A dione of formula V can be prepared by oxydation of a ketone of formula IV, in which Y is O, C=Z is CH₂, and R³ and X are as defined hereinabove, e.g. by treatment of a compound IV with selenium dioxide in dioxane at 100° C.

A ketone of formula IV, in which Y is O, C=Z is CH₂, and R³ and X are as defined hereinabove, can be prepared by, firstly, reacting a pyridine of formula II in which R′ is COOCH₂CH₃ and X is as defined hereinabove with a base, such as lithium diisopropylamide, in an inert solvent, such as tetrahydrofuran, at a temperature of −50 to −80° C., and thereafter, adding to the reaction mixture a methyl ester of formula III in which n is 1 and R³ is as defined hereinabove, and subsequently allowing the reaction temperature to warm up to 20° C. The COOCH₃ group in the obtained reaction intermediate is cleaved and the resulting carboxylic acid group concomitantly decarboxylated by heating with conc. hydrochloric acid to 110° C. for 5 to 10 h.

Procedure B: Compounds of formula I can also be obtained by replacing in the procedure for the preparation of VII, given above, ammonium acetate by an amine of formula IX, in which R¹ is as defined hereinabove. As it is the case for procedure A, this generally known method provides a mixture of 2 regioisomeric products, and the compound of formula I can be isolated in pure form by chromatography or crystallization of the crude reaction product.

Procedure C: Alternatively, compounds of formula I can be prepared in a regiospecific manner by heating an oxime of formula X together with an aldehyde of formula VI and an amine of formula IX in acetic acid to 100-110° C. for 1 to 10 h, followed by reduction of the resulting imidazole-oxide of formula XI, e.g. with phosphorous trichloride in dichloromethane at 20° C.

An oxime of the formula X can be prepared by nitrosation of a ketone of formula IV, in which C═Y is CH₂, Z is O, and R³ and X are as defined hereinabove, e.g using sodium nitrite in aqueous acetic acid. It can be obtained and used in the preparation of XI as a single isomer or as a mixture of (E)- and (Z)-oximes.

A ketone of formula IV, in which C═Y is CH₂, Z is O, and R³ and X are as defined hereinabove, can be prepared by, firstly, reacting a pyridine of formula II in which R′ is CH₃ and X is as defined hereinabove with a base, such as lithium diisopropylamide, in an inert solvent, such as tetrahydrofuran, at a temperature of −50 to −80° C., and thereafter, adding to the reaction mixture a methyl ester of formula III in which n is 0 and R³ is as defined hereinabove, and subsequently allowing the reaction temperature to warm up to 20° C.

A product of formula I, in which X is OCH₃, can also be prepared by heating a compound of formula I, in which X is Cl, with potassium hydroxide in a mixture of methanol and DMSO to 50 to 80° C.

The detailed conditions for the preparation of representative compounds of general formula I can be found in the examples hereinafter.

The pharmaceutically acceptable salts can be manufactured readily according to methods known per se and taking into consideration the nature of the compound to be converted into a salt. Inorganic or organic acids such as, for example, hydrochloric acid, hydrobromic acid, sulphuric acid, nitric acid, phosphoric acid or citric acid, formic acid, fumaric acid, maleic acid, acetic acid, succinic acid, tartaric acid, methanesulphonic acid, p-toluenesulphonic acid and the like are suitable for the formation of pharmaceutically acceptable salts of basic compounds of formula I.

The present invention also provides pharmaceutical compositions containing compounds of the invention, for example compounds of formula I and their pharmaceutically acceptable acid addition salts, and a pharmaceutically acceptable carrier. Such pharmaceutical compositions can be in the form of tablets, coated tablets, dragées, hard and soft gelatine capsules, solutions, emulsions or suspensions. The pharmaceutical compositions also can be in the form of suppositories or injectable solutions.

The pharmaceutical compounds of the invention, in addition to one or more compounds of the invention, contain a pharmaceutically acceptable carrier. Suitable pharmaceutically acceptable carriers include pharmaceutically inert, inorganic and organic carriers. Lactose, corn starch or derivatives thereof, talc, stearic acids or its salts and the like can be used, for example, as such carriers for tablets, coated tablets, dragées and hard gelatine capsules. Suitable carriers for soft gelatine capsules are, for example, vegetable oils, waxes, fats, semi-solid and liquid polyols and the like. Depending on the nature of the active substance no carriers are, however, usually required in the case of soft gelatine capsules. Suitable carriers for the production of solutions and syrups are, for example, water, polyols, glycerol, vegetable oil and the like. Suitable carriers for suppositories are, for example, natural or hardened oils, waxes, fats, semi-liquid or liquid polyols and the like.

In addition, the pharmaceutical compositions can contain preservatives, solubilizers, stabilizers, wetting agents, emulsifiers, sweeteners, colorants, flavorants, salts for varying the osmotic pressure, buffers, masking agents or antioxidants. They can also contain still other therapeutically valuable substances.

As mentioned earlier, medicaments containing a compound of formula I or a pharmaceutically acceptable salt thereof and a therapeutically inert excipient are also an object of the present invention, as is a process for the production of such pharmaceutical compositions which comprises bringing one or more compounds of formula I or pharmaceutically acceptable salts thereof and, if desired, one or more other therapeutically valuable substances into a galenical dosage form together with one or more therapeutically inert carriers.

The compounds of formula I and their pharmaceutically acceptable salts are metabotropic glutamate receptor antagonists and can be used for the treatment or prevention of acute and/or chronic neurological disorders, such as psychosis, schizophrenia, Alzheimer's disease, cognitive disorders and memory deficits. Other treatable indications are restricted brain function caused by bypass operations or transplants, poor blood supply to the brain, spinal cord injuries, head injuries, hypoxia caused by pregnancy, cardiac arrest and hypoglycaemia. Further treatable indications are acute and chronic pain, Huntington's chorea, ALS, dementia caused by AIDS, eye injuries, retinopathy, idiopathic parkinsonism or parkinsonism caused by medicaments as well as conditions which lead to glutamate-deficient functions, such as e.g. muscle spasms, convulsions, migraine, urinary incontinence, nicotine addiction, psychoses, opiate addiction, anxiety, vomiting, dyskinesia, depression and glioma.

The dosage at which compounds of the invention can be administered can vary within wide limits and will, of course, be fitted to the individual requirements in each particular case. In general, the effective dosage for oral or parenteral administration is between 0.01-20 mg/kg/day, with a dosage of 0.1-10 mg/kg/day being preferred for all of the indications described. The daily dosage for an adult human being weighing 70 kg accordingly lies between 0.7-1400 mg per day, preferably between 7 and 700 mg per day.

The compounds of the present invention are group II mGlu receptor antagonists. The compounds show activities, as measured in the assay described below, of 0.800 μM or less, typically 0.100 μM or less, and ideally of 0.030 μM or less. In the table below are described some specific Ki values of representative compounds.

| | Ex. No. | | | | | | | |
|---|---|---|---|---|---|---|---|---|
| | 3 | 11 | 19 | 26 | 27 | 37 | 43 | 47 |
| Kᵢ mGlu2 (μM) | 0.007 | 0.013 | 0.034 | 0.063 | 0.021 | 0.049 | 0.111 | 0.018 |

[³H]-LY354740 Binding on mGlu2 Transfected CHO Cell Membranes.
Transfection and Cell Culture

cDNA encoding the rat mGlu2 receptor protein in pBluescript II was subcloned into the eukaryotic expression vector pcDNA I-amp from Invitrogen Ltd (Paisley, UK). This vector construct (pcD1mGR²) was co-transfected with a psvNeo plasmid encoding the gene for neomycin resistance, into CHO cells by a modified calcium phosphate method described by Chen & Okayama (1988). The cells were maintained in Dulbecco's Modified Eagle medium with reduced L-glutamine (2 mM final concentration) and 10% dialysed foetal calf serum from Gibco-Invitrogen (Carlsbad, Calif., USA). Selection was made in the presence of G-418 (1000 ug/ml final) and MCPG??. Clones were identified by reverse transcription of 5 μg total RNA, followed by PCR using mGlu2 receptor specific primers 5′-atcactgcttgggtttctggcactg-3′ and 540 -agcatcactgtgggtggcataggagc-3′ in 60 mM Tris HCl (pH 10), 15 mM (NH4)₂SO₄, 2 mM MgCl₂, 25 units/ml Taq Polymerase with 30 cycles annealing at 60° C. for 1 min., extention at 72° C. for 30 s, and 1 min. 95° C. denaturation.

Membrane Preparation

Cells, cultured as above, were harvested and washed three times with cold PBS and frozen at −80° C. The pellet was resuspended in cold 20 mM HEPES-NaOH buffer containing 10 mM EDTA (pH 7.4), and homogenised with a polytron (Kinematica, AG, Littau, Switzerland) for 10 s at 10 000 rpm. After centrifugation for 30 min. at 4° C., the pellet was washed once with the same buffer, and once with cold 20 mM HEPES-NaOH buffer containing 0.1 mM EDTA, (pH 7.4). Protein content was measured using the micro BCA method from Pierce-Perbio (Rockford, Ill., USA) using bovine serum albumin as standard.

[³H]-LY354740 Binding

After thawing, the membranes were resuspended in cold 50 mM Tris-HCl buffer containing 2 mM MgCl₂ (pH 7) (binding buffer). The final concentration of the membranes in the assays was 25 μg protein/ml. Inhibition experiments were performed with membranes incubated with 10 nM [³H]-LY354740 at room temperature, for 1 hour, in presence of various concentrations of the compound to be tested. Following the incubations, membranes were filtered onto Whatmann GF/B glass fiber filters and washed 5 times with cold binding buffer. Non specific binding was measured in the presence of 10 μM DCG IV. After transfer of the filters into plastic vials containing 10 ml of Ultima-gold scintillation fluid from Perkin-Elmer (Boston, Mass., USA), the radioactivity was measured by liquid scintillation in a Tri-Carb 2500 TR counter (Packard, Zürich, Switzerland).

Data Analysis.

The inhibition curves were fitted with a four parameter logistic equation giving IC₅₀ values, and Hill coefficients.

### EXAMPLES

Almost all of the starting materials used in the general scheme 1 and procedures A, B and C is commercially available. The starting materials which was not commercially available have been prepared according to the procedures as outlined in the examples hereafter and unless otherwise specified, the intermediate compounds described therein are novel compounds.

### Example 1

### 4-[2-(2,6-Dichloro-phenyl)-1-methyl-5-(4-methyl-phenyl)-1H-imidazol-4-yl]-pyridine

Sodium hydride (16 mg, 0.65 mmol) was added at 0° C. to a solution of 4-[2-(2,6-dichloro-phenyl)-5-(4-methyl-phenyl)-1H-imidazol-4-yl]-pyridine (190 mg, 0.5 mmol) and methyl iodide (0.083 mL, 0.65 mmol) in DMF (0.7 mL). The mixture was stirred for 4 h and then kept for 20 h at 0° C. Ethyl acetate (10 mL) was added and the mixture was washed with 10% aqueous sodium chloride solution. The organic layer was dried over sodium sulfate and evaporated in vacuo. The residue was chromatographed on silica gel using ethyl acetate/hexane (2:1, v/v) as eluent. The more polar methylation product was collected and crystallized from ethyl acetate/hexane to give the title compound (55 mg) as white solid. MS (ISP) 394.2 [(M+H)⁺]; mp 214-217° C.

The starting material was prepared in the following way:

### 1-A: 1-Pyridin-4-yl-2-(4-methyl-phenyl)-ethane-1,2-dione

A mixture of 1-pyridin-4-yl-2-(4-methyl-phenyl)-ethanone (6.8 g, 32 mmol) and selenium dioxide (5.53 g, 50 mmol) in dioxane (100 mL) was refluxed for 2 h. The mixture was cooled and insoluble material was removed by filtration. The solution was evaporated in vacuo and the residue was chromatographed on silica gel using ethyl acetate/hexane (1:9 to 1:0.25, v/v) as eluent. The product was crystallized from hexane to give the title compound (4.2 g) as yellow solid. Mp 65° C.

### 1-B: 4-[2-(2,6-Dichloro-phenyl)-5-(4-methyl-phenyl)-1H-imidazol-4-yl]-pyridine

A mixture of 1-pyridin-4-yl-2-(4-methyl-phenyl)-ethane-1,2-dione (0.90 g, 4.0 mmol), 2,6-dichloro-benzaldehyde (0.70 g, 4.0 mmol), ammonium acetate (3.1 g, 40 mmol), and acetic acid (10 mL) was heated to 100° C. for 4 h. The cooled reaction mixture was stirred with ice-cold 12% aqueous ammonia (40 mL) for 20 min and the mixture extracted with ethyl acetate (150 mL). The organic layer was washed with brine, dried over sodium sulfate and evaporated in vacuo. The residue was crystallized from ethyl acetate/hexane to give the title compound (1.04 g) as pale-yellow solid. Mp 295-296° C.

### Example 2

### 4-[1-Methyl-5-(4-methyl-phenyl)-2-(2,4,6-trimethyl-phenyl)-1H-imidazol-4-yl]-pyridine

4-[5-(4-Methyl-phenyl)-2-(2,4,6-trimethyl-phenyl)-1H-imidazol-4-yl]-pyridine (177 mg) was subjected in an analogous manner to the procedure described in Example 1 to give the title compound (70 mg) as a white solid. MS (ISP) 368.4 [(M+H)⁺]; mp 204-207° C. The starting material was prepared in the following way:

### 2-A: 4-[5-(4-Methyl-phenyl)-2-(2,4,6-trimethyl-phenyl)-1H-imidazol-4-yl]-pyridine

A mixture of 1-pyridin-4-yl-2-(4-methyl-phenyl)-ethane-1,2-dione (0.9 g, 4.0 mmol), 2,4,6-trimethyl-benzaldehyde (0.59 g, 4.0 mmol), ammonium acetate (3.1 g, 40 mmol), and acetic acid (10 mL) was heated to 100° C. for 4 h. The cooled reaction mixture was stirred with ice-cold 10% aqueous ammonia (60 mL) for 20 min and the mixture extracted with ethyl acetate (150 mL). The organic layer was washed with brine, dried over sodium sulfate and evaporated in vacuo. The residue was crystallized from ethyl acetate/hexane to give the title compound (0.99 g) as pale-yellow solid. Mp 277-279° C.

### Example 3

### 4-[5-(4-Chloro-phenyl)-2-(2,6-dichloro-phenyl)-1-methyl-1H-imidazol-4-yl]-pyridine

4-[5-(4-Chloro-phenyl)-2-(2,6-dichloro-phenyl)-imidazol-4-yl]-pyridine (200 mg) was subjected in an analogous manner to the procedure described in Example 1 to give the title compound (33 mg) as a white solid. MS (ISP) 414.2 [(M+H)⁺]; mp 257-260° C.

### Example 4

### 4-[1-Ethyl-5-(4-methyl-phenyl)-2-(2,4,6-trimethyl-phenyl)-1H-imidazol-4-yl]-pyridine

Sodium hydride (16 mg, 0.65 mmol) was added at 0° C. to a solution of 4-[5-(4-methyl-phenyl)-2-(2,4,6-trimethyl-phenyl)-1H-imidazol-4-yl]-pyridine (177 mg, 0.5 mmol) and ethyl bromide (0.049 mL, 0.65 mmol) in DMF (0.7 mL). After being stirred for 4 h at 0° C., the mixture was diluted with ethyl acetate (20 mL) and washed with 10% aqueous sodium chloride solution. The organic layer was dried over sodium sulfate and evaporated in vacuo. The residue was crystallized from ethyl acetate/hexane to give the title compound (52 mg) as white solid. MS (ISP) 382.4 [(M+H)⁺]; mp 137-140° C.

### Example 5

### 4-[5-(4-Chloro-phenyl)-2-(2,6-dichloro-phenyl)-1-ethyl-1H-imidazol-4-yl]-pyridine

4-[5-(4-Chloro-phenyl)-2-(2,6-dichloro-phenyl)-imidazol-4-yl]-pyridine (100 mg) was subjected in an analogous manner to the procedure described in Example 4. The crude product was chromatographed on silica gel using ethyl acetate/hexane (2:1, v/v) as eluent and the more polar ethylation product was collected and crystallized from diethyl ether to give the title compound (33 mg) as white solid. MS (ISP) 428.3 [(M+H)⁺]; mp 193-196° C.

### Example 6

### 4-[1-Ethyl-5-(2,4-dichloro-phenyl)-2-(2,4,6-trimethyl-phenyl)-1H-imidazol-4-yl]-pyridine

4-[5-(2,4-Dichloro-phenyl)-2-(2,4,6-trimethyl-phenyl)-1H-imidazol-4-yl]-pyridine (163 mg) was subjected in an analogous manner to the procedure described in Example 4 to give the title compound (60 mg) as pale-yellow solid. MS (ISP) 436.3 [(M+H)⁺]; mp 191-193° C. The starting material was prepared in the following way:

### 6-A: 2-(2,4-Dichlorophenyl)-1-pyridin-4-yl-1-ethanone

To a 1 N solution of lithium diisopropylamide in tetrahedrofuran (0.15 L, 0.15 mol) was added over 20 min. at −60° C. a solution of ethyl 2,4-dichloro-benzeneacetate (35.0 g, 0.15 mol) in tetrahydrofuran (0.075 L) followed by the addition of a solution of ethyl 4-pyridinecarboxylate (20.6 g, 0.15 mol) in tetrahydrofuran (0.075 L). The reaction mixture was stirred at 20° C. for 20 h and then evaporated in vacuo. Ice-water (0.15 L) and 1 N hydrochloric acid (0.15 L) were added to the remaining oil and the mixture was extracted with dichloromethane. The organic layer was washed with brine, dried over sodium sulfate, and evaporated in vacuo. The residual oil was suspended in conc. hydrochloric acid (0.1 L) and the mixture was heated to 110° C. for 6 h. The mixture was poured into ice-water, the pH was set to 8 by the addition of sodium bicarbonate, and the product was subsequently extracted with dichloromethane. The organic layer was washed with brine, dried over sodium sulfate and evaporated in vacuo. The residue was chromatographed on silica gel using ethyl acetate/hexane (1:9 to 4:1) as eluent and the purified product was crystallized from diisopropyl ether to give the title compound (5.7 g). Mp 77° C.

### 6-B: 1-(2,4-Dichloro-phenyl)-2-pyridin-4-yl-ethane-1,2-dione

2-(2,4-Dichlorophenyl)-1-pyridin-4-yl-1-ethanone (5.0 g) was oxidized with selenium dioxide in an analogous manner as described in Example 1-A to give the title compound (2.6 g) as yellow solid. Mp 85° C.

### 6-C: 4-[5-(2,4-Dichloro-phenyl)-2-(2,4,6-trimethyl-phenyl)-1H-imidazol-4-yl]-pyridine

1-(2,4-Dichloro-phenyl)-2-pyridin-4-yl-ethane-1,2-dione (0.56 g) was subjected in an analogous manner to the procedure described in Example 1-B to give the title compound (0.40 g) as pale yellow solid. Mp 276-277° C.

### Example 7

### 4-[5-(4-tert-Butyl-phenyl)-1-ethyl-2-(2,4,6-trimethyl-phenyl)-1H-imidazol-4-yl]-pyridine

4-[5-(4-tert-Butyl-phenyl)-2-(2,4,6-trimethyl-phenyl)-1H-imidazol-4-yl]-pyridine (158 mg) was subjected in an analogous manner to the procedure described in Example 4. The crude product was chromatographed on silica gel using ethyl acetate/hexane (2:1, v/v) as eluent and the more polar ethylation product was collected and crystallized from diethyl ether to give the title compound (94 mg) as white foam. MS (ISP) 424.5 [(M+H)⁺]; mp 202-205° C. The starting material was prepared in the following way:

### 7-A: 1-(4-tert-Butyl-phenyl)-2-pyridin-4-yl-ethane-1,2-dione

2-(4-tert.-Butyl-phenyl)-1-(pyridin-4-yl)-1-ethanone (4.3 g) was oxidized with selenium dioxide in an analogous manner as described in Example 1-A to give the title compound (8.5 g) as yellow oil. Bp 180° C./0.3 mbar.

### 7-B: 4-[5-(4-tert-Butyl-phenyl)-2-(2,4,6-trimethyl-phenyl)-1H-imidazol-4-yl]-pyridine

1-(4-tert-Butyl-phenyl)-2-pyridin-4-yl-ethane-1,2-dione (0.53 g) was subjected in an analogous manner to the procedure described in Example 2-A to give the title compound (0.63 g) as pale yellow solid, mp 303-304° C.

### Example 8

### 4-[5-(4-Chloro-phenyl)-1-ethyl-2-(2-methyl-phenyl)-1H-imidazol-4-yl]-pyridine

4-[5-(4-Chloro-phenyl)-2-(2-methyl-phenyl)-1H-imidazol-4-yl]-pyridine (138 mg) was subjected in an analogous manner to the procedure described in Example 4. The crude product was chromatographed on silica gel using ethyl acetate/hexane (2:1, v/v) as eluent and the more polar ethylation product was collected and crystallized from ethyl acetate/hexane to give the title compound (24 mg) as white solid. MS (ISP) 374.4 [(M+H)⁺]; mp 173-175° C. The starting material was prepared in the following way:

### 8-A: 4-[5-(4-Chloro-phenyl)-2-(2-methyl-phenyl)-1H-imidazol-4-yl]-pyridine

1-(4-Chloro-phenyl)-2-pyridin-4-yl-ethane-1,2-dione (0.37 g) and 2-methyl-benzaldehyde were subjected in an analogous manner to the procedure described in Example 1-B to give the title compound (0.27 g) as pale yellow solid. Mp 211-213° C.

### Example 9

### 4-[5-(4-Chloro-phenyl)-1-ethyl-2-phenyl-1H-imidazol-4-yl]-pyridine

4-(4-Chloro-phenyl)-2-phenyl-5-pyridin-4-yl-imidazole (133 mg) was subjected in an analogous manner to the procedure described in Example 4. The crude product was chromatographed on silica gel using ethyl acetate/hexane (2:1, v/v) as eluent and the more polar ethylation product was collected to give the title compound (14 mg) as white foam. MS (ISP) 360.2 [(M+H)⁺]; mp 267-270° C.

### Example 10

### 4-[1-Ethyl-5-(4-fluoro-phenyl)-2-(2,4,6-trimethyl-phenyl)-1H-imidazol-4-yl]-pyridine

4-[4-(4-Fluoro-phenyl)-2-(2,4,6-trimethyl-phenyl)-imidazol-5-yl]-pyridine (71 mg) was subjected in an analogous manner to the procedure described in Example 4. The crude product was chromatographed on silica gel using ethyl acetate/hexane (2:1, v/v) as eluent and the more polar ethylation product was collected and crystallized from ethyl acetate/hexane to give the title compound (41 mg) as white solid. MS (ISP) 386.3 [(M+H)⁺]; mp 194-196° C.

### Example 11

### 4-[2-(2,6-Dichloro-phenyl)-1-ethyl-5-(4-methyl-phenyl)-1H-imidazol-4-yl]-pyridine

4-[2-(2,6-Dichloro-phenyl)-5-(4-methyl-phenyl)-1H-imidazol-4-yl]-pyridine (114 mg) was subjected in an analogous manner to the procedure described in Example 4 to give the title compound (88 mg) as white solid. MS (ISP) 408.2 [(M+H)⁺]; mp 191-193° C.

### Example 12

### 4-[5-(4-Chloro-phenyl)-1-ethyl-2-(2,4,6-trimethyl-phenyl)-1H-imidazol-4-yl]-pyridine

4-[5-(4-Chloro-phenyl)-2-(2,4,6-trimethyl-phenyl)-imidazol-4-yl]-pyridine (112 mg) was subjected in an analogous manner to the procedure described in Example 4. The crude product was chromatographed on silica gel using ethyl acetate/hexane (2:1, v/v) as eluent and the more polar ethylation product was collected and crystallized from diethyl ether/hexane to give the title compound (70 mg) as white solid. MS (ISP) 402.3 [(M+H)⁺]; mp 226-227° C.

### Example 13

### 4-[1-Ethyl-5-(4-trifluoromethyl-phenyl)-2-(2,4,6-trimethyl-phenyl)-1H-imidazol-4-yl]-pyridine

4-[4-(4-Trifluoromethyl-phenyl)-2-(2,4,6-trimethyl-phenyl)-imidazol-5-yl]-pyridine (81 mg) was subjected in an analogous manner to the procedure described in Example 4. The crude product was chromatographed on silica gel using ethyl acetate/hexane (2:1, v/v) as eluent and the more polar ethylation product was collected and crystallized from ethyl acetate/hexane to give the title compound (40 mg) as white solid. MS (ISP) 436.4 [(M+H)⁺]; mp 227-228° C. The starting material was prepared in the following way:

### 13-A: 1-Pyridin-4-yl-2-(4-trifluoromethyl-phenyl)-ethane-1,2-dione

Pyridin-4-yl-2-(4-trifluoromethyl-phenyl)-ethanone was oxidized with selenium dioxide in an analogous manner as described in Example 1-A to give the title compound as yellow solid. Mp 70-72° C.

### 13-B: 4-[4-(4-Trifluoromethyl-phenyl)-2-(2,4,6-trimethyl-phenyl)-imidazol-5-yl]-pyridine

Pyridin-4-yl-2-(4-trifluoromethyl-phenyl)-ethane-1,2-dione was subjected in an analogous manner to the procedure described in Example 1-B to give the title compound as pale yellow solid. Mp>260° C.

### Example 14

### 4-[5-(4-Chloro-phenyl)-2-(2,6-dimethyl-phenyl)-1-ethyl-1H-imidazol-4-yl]-pyridine

4-[5-(4-Chloro-phenyl)-2-(2,6-dimethyl-phenyl)-imidazol-4-yl]-pyridine (72 mg subjected in an analogous manner to the procedure described in Example 4. The crude product was chromatographed on silica gel using ethyl acetate/hexane (2:1, v/v) as eluent and the more polar ethylation product was collected and crystallized from ethyl acetate/hexane to give the title compound (43 mg) as white solid. MS (ISP) 388.2 [(M+H)⁺]; mp 171-173° C.

### Example 15

### 4-[5-(4-Chloro-phenyl)-2-(2-chloro-phenyl)-1-ethyl-1H-imidazol-4-yl]-pyridine

4-[2-(2-Chloro-phenyl)-5-(4-chloro-phenyl)-1H-imidazol-4-yl)-pyridine (73 mg) was subjected in an analogous manner to the procedure described in Example 4. The crude product was chromatographed on silica gel using ethyl acetate/hexane (2:1, v/v) as eluent and the more polar ethylation product was collected and crystallized from ethyl acetate/hexane to give the title compound (32 mg) as white solid. MS (ISP) 394.2 [(M+H)⁺]; mp 144-147° C.

### Example 16

### 4-[2-(2-Bromo-phenyl)-5-(4-chloro-phenyl)-1-ethyl-1H-imidazol-4-yl]-pyridine

4-[2-(2-Bromo-phenyl)-4-(4-chloro-phenyl)-imidazol-5-yl]-pyridine (82 mg) was subjected in an analogous manner to the procedure described in Example 4. The crude product was chromatographed on silica gel using ethyl acetate/hexane (2:1, v/v) as eluent and the more polar ethylation product was collected and crystallized from ethyl acetate/hexane to give the title compound (50 mg) as white solid. MS (ISP) 438.2 [(M+H)⁺]; mp 144-147° C.

### Example 17

### 4-[2-(2-Chloro-6-methyl-phenyl)-5-(4-chloro-phenyl)-1-ethyl-1H-imidazol-4-yl]-pyridine

4-[2-(2-Chloro-6-methyl-phenyl)-4-(4-chloro-phenyl)-imidazol-5-yl]-pyridine (76 mg) was subjected in an analogous manner to the procedure described in Example 4. The crude product was chromatographed on silica gel using ethyl acetate/hexane (2:1, v/v) as eluent and the more polar ethylation product was collected and crystallized from ethyl acetate/hexane to give the title compound (49 mg) as white solid. MS (ISP) 408.2 [(M+H)⁺]; mp 188-190° C.

### Example 18

### 4-[5-(4-Chloro-phenyl)-1-ethyl-2-(2,4,6-trimethoxy-phenyl)-1H-imidazol-4-yl]-pyridine

4-[5-(4-Chloro-phenyl)-2-(2,4,6-trimethoxy-phenyl)-1H-imidazol-4-yl]-pyridine (84 mg) was subjected in an analogous manner to the procedure described in Example 4. The crude product was chromatographed on silica gel using ethyl acetate/hexane (2:1) as eluent and the more polar ethylation product was collected and crystallized from ethyl acetate/hexane to give the title compound (45 mg) as white solid. MS (ISP) 450.3 [(M+H)⁺]; mp 255-256° C.

### Example 19

### 4-[1-Allyl-2-(2,6-dichloro-phenyl)-5-(4-methyl-phenyl)-1H-imidazol-4-yl]-pyridine

Sodium hydride (31 mg, 1.3 mmol) was added at 0° C. to a solution of 4-[2-(2,6-dichloro-phenyl)-5-(4-methyl-phenyl)-1H-imidazol-4-yl]-pyridine (380 mg, 1.0 mmol) and allyl bromide (0.11 mL, 1.3 mmol) in DMF (1.4 mL). The mixture was stirred for 7 h, and then kept for 16 h at 0° C. Ethyl acetate (20 mL) was added and the mixture was washed with 10% aqueous sodium chloride solution. The organic layer was dried over sodium sulfate and evaporated in vacuo. The residue was chromatographed on silica gel using ethyl acetate/hexane (2:1, v/v) as eluent. The more polar allylation product was collected and crystallized from ethyl acetate/hexane to give the title compound (178 mg) as white solid. MS (ISP) 420.2 [(M+H)⁺]; mp 147-149° C.

### Example 20

### 4-[1-Allyl-5-(4-trifluoromethyl-phenyl)-2-(2,4,6-trimethyl-phenyl)-1H-imidazol-4-yl]-pyridine

4-[4-(4-Trifluoromethyl-phenyl)-2-(2,4,6-trimethyl-phenyl)-imidazol-5-yl]-pyridine (81 mg) was subjected in an analogous manner to the procedure described in Example 19 to give the title compound (26 mg) as white solid. MS (ISP) 448.3 [(M+H)⁺]; mp 209-210° C.

### Example 21

### 4-[1-Allyl-5-(4-chloro-phenyl)-2-(2,4,6-trimethyl-phenyl)-1H-imidazol-4-yl]-pyridine

4-[5-(4-Chloro-phenyl)-2-(2,4,6-trimethyl-phenyl)-imidazol-4-yl]-pyridine (373 mg) was subjected in an analogous manner to the procedure described in Example 19 to give the title compound (39 mg) as white solid. MS (ISP) 414.2 [(M+H)⁺]; mp 151-154° C.

### Example 22

### 4-[1-Allyl-5-(4-chloro-phenyl)-2-(2,6-dichloro-phenyl)-1H-imidazol-4-yl]-pyridine

4-[5-(4-Chloro-phenyl)-2-(2,6-dichloro-phenyl)-imidazol-4-yl]-pyridine (401 mg) was subjected in an analogous manner to the procedure described in Example 19 to give the title compound (56 mg) as white solid. MS (ISP) 440.3 [(M+H)⁺]; mp 163-165° C.

### Example 23

### 4-[1-Allyl-5-(4-methyl-phenyl)-2-(2,4,6-trimethyl-phenyl)-1H-imidazol-4-yl]-pyridine

4-[5-(4-methyl-phenyl)-2-(2,4,6-trimethyl-phenyl)-1H-imidazol-4-yl]-pyridine (353 mg) was subjected in an analogous manner to the procedure described in Example 19 to give the title compound (90 mg) as white solid. MS (ISP) 394.5 [(M+H)⁺]; mp 115-118° C.

### Example 24

### 4-[5-(4-Chloro-phenyl)-1-cyclopentyl-2-(2,6-dichloro-phenyl)-1H-imidazol-4-yl]-pyridine

Sodium hydride (24 mg, 1.0 mmol) was added at 0° C. to a solution of 4-[5-(4-chloro-phenyl)-2-(2,6-dichloro-phenyl)-imidazol-4-yl]-pyridine (200 mg, 0.5 mmol) and methanesulfonic acid cyclopentyl ester (164 mg, 1.0 mmol) in DMF (2 mL). The mixture was stirred for 24 h at 85° C. Ethyl acetate (20 mL) was added and the mixture was washed with 10% aqueous sodium chloride solution. The organic layer was dried over sodium sulfate and evaporated in vacuo. The residue was chromatographed on silica gel using dichloromethane/methanol/acetic acid (95:4:1, v/v/v) as eluent. The more polar product was collected and crystallized from ethyl acetate/hexane to give the title compound (23 mg) as white solid. MS (ISP) 468.1 [(M+H)⁺].

### Example 25

### 4-[5-(4-Chloro-phenyl)-2-(2,6-dichloro-phenyl)-1-(2,2,2-trifluoro-ethyl)-1H-imidazol-4-yl]-pyridine

A mixture of 1-(4-chloro-phenyl)-2-pyridin-4-yl-ethane-1,2-dione (122 mg, 0.5 mmol), 2,6-dichloro-benzaldehyde (88 mg, 0.5 mmol), 2,2,2-trifluoroethylamine hydrochloride (339 mg, 2.5 mmol), ammonium acetate (192 mg, 2.5 mmol), and acetic acid (2.5 mL), was heated to 100° C. for 4 h. The cooled reaction mixture was diluted with water and then extracted with ethyl acetate (40 mL). The organic layer was washed with brine, dried over sodium sulfate and evaporated in vacuo. The residue was chromatographed on silica gel using dichloromethane/methanol/acetic acid (95:4:1, v/v/v) as eluent. The more polar product was collected and crystallized from ethyl acetate/hexane to give the title compound (57 mg) as white solid. MS (ISP) 482.2 [(M+H)⁺]; mp 213-214° C.

### Example 26

### 2-[5-(4-Chloro-phenyl)-4-pyridin-4-yl-2-(2,4,6-trimethyl-phenyl)-imidazol-1-yl]-ethanol

1-(4-Chloro-phenyl)-2-pyridin-4-yl-ethane-1,2-dione (122 mg, 0.5 mmol) was subjected to the procedure described in Example 25, but using 2,4,6-trimethyl-benzaldehyde (74 mg, 0.5 mmol) instead of 2,6-dichloro-benzaldehyde, and ethanolamine (0.15 mL, 2.5 mmol) instead of 2,2,2-trifluoroethylamine hydrochloride. The crude product was chromatographed on silica gel using dichloromethane/methanol/acetic acid (95:4:1, v/v/v) as eluent. The more polar product was collected and stirred with methanol (1 mL)/2 N NaOH (0.2 mL) for 1 h at 20° C. The mixture was partitioned between ethyl acetate and water, the organic layer was dried over sodium sulfate and evaporated in vacuo to give the title compound (8 mg) as white solid. MS (ISP) 418.4 [(M+H)⁺].

### Example 27

### 4-[5-(4-Chloro-phenyl)-1-cyclopropyl-2-(2,6-dichloro-phenyl)-1H-imidazol-4-yl]-pyridine

A mixture of 1-(4-chloro-phenyl)-2-pyridin-4-yl-ethane-1,2-dione 2-oxime (3.92 g, 15 mmol), 2,6-dichloro-benzaldehyde (2.63, 15 mmol), cyclopropylamine (3.15 mL, 45 mmol), and acetic acid (50 mL), was heated to 100° C. for 5 h. The reaction mixture was cooled and the pH was set to 9 by the addition of 25% aqueous ammonia. The mixture was extracted with ethyl acetate (200 mL). The organic layer was washed with sat. sodium carbonate solution (50 mL) and with water 50 mL), dried over sodium sulfate, and evaporated in vacuo. The residue was triturated with ethyl acetate (30 mL) and the solid material (5.48 g) was collected by filtration and stirred for 2 h at 20° C. with phosphorous trichloride (1.05 mL, 12 mmol) in chloroform (40 mL). The solution was diluted with dichloromethane, washed with sat sodium carbonate solution and with brine, dried over sodium sulfate and evaporated in vacuo. The residue was crystallized from ethyl acetate/hexane to give the title compound (4.78 g) as white solid. MS (ISP) 440.2 [(M+H)⁺]; mp 210-211° C.

The starting material was prepared in the following way:

### 27-A: 1-(4-Chloro-phenyl)-2-pyridin-4-yl-ethane-1,2-dione 2-oxime

To a solution of 1-(4-chloro-phenyl)-2-pyridin-4-yl-ethanone (1.16 g, 5.0 mmol) in acetic acid (20 mL) was added dropwise at 5-8° C. a solution of sodium nitrite (0.40 g, 5.8 mmol) in water (4 mL), a thick precipitate being formed. The mixture was stirred for 30 min at 20° C. Water (20 mL) was added and stirring was continued for 15 min at 0° C. The precipitate was isolated by filtration and dried to give the title compound (1.18 g) as off-white solid. MS (ISP) 261.0 [(M+H)⁺].

### Example 28

### 4-[5-(4-Chloro-phenyl)-2-(2,6-dichloro-phenyl)-1-prop-2-ynyl-1H-imidazol-4-yl]-pyridine

1-(4-Chloro-phenyl)-2-pyridin-4-yl-ethane-1,2-dione2-oxime (260 mg, 1.0 mmol) was subjected in an analogous manner to the procedure described in Example 27, but using propargylamine instead of cyclopropylamine, to give the title compound (48 mg) as white solid. Mp 169-172° C.

### Example 29

### 3-[5-(4-Chloro-phenyl)-2-(2,6-dichloro-phenyl)-4-pyridin-4-yl-imidazol-1-yl]-propan-1-ol

1-(4-Chloro-phenyl)-2-pyridin-4-yl-ethane-1,2-dione2-oxime (261 mg, 1 mmol) was subjected in an analogous manner to the procedure described in Example 27, but using 3-amino-1-propanol instead of cyclopropylamine. The resulting ester was cleaved with methanol/2 N NaOH as described in Example 26 to give the title compound (234 mg) as white solid. MS (ISP) 458.2 [(M+H)⁺]; mp 233-235° C.

### Example 30

### 4-[2-(2,4-Bis-trifluoromethyl-phenyl)-5-(4-chloro-phenyl)-1-ethyl-1H-imidazol-4-yl]-pyridine

A mixture of 1-(4-chloro-phenyl)-2-pyridin-4-yl-ethane-1,2-dione2-oxime (291 mg, 1 mmol), bis-2,4-(trifluoromethyl)-benzaldehyde (290, 1.2 mmol), 70% aqueous ethylamine (0.4 mL, 5.0 mmol), and acetic acid (4 mL) was heated to 110° C. for 16 h. The reaction mixture was cooled and the pH was set to 6 by the addition of 25% aqueous ammonia. The mixture was extracted with ethyl acetate (20 mL), and the organic layer was washed with sat. sodium carbonate solution (10 mL) and with water 10 mL), dried over sodium sulfate, and evaporated in vacuo. The residue was chromatographed on silica gel using ethyl dichloromethane/methanol (20:1, v/v) as eluent to give the product N-oxide (398 mg) as white solid. This material was stirred for 2 h at 20° C. with phosphorous trichloride (0.5 mL, 5.7 mmol) in dichloromethane (5 mL). The solution was diluted with dichloromethane, washed with sat. sodium carbonate solution and with brine, dried over sodium sulfate, and evaporated in vacuo. The residue was crystallized from ethyl acetate/hexane to give the title compound (290 mg) as white solid. MS (ISP) 496.1 [(M+H)⁺]; mp 148-150° C.

### Example 31

### 3-[5-(4-Chloro-phenyl)-1-ethyl-4-pyridin-4-yl-1H-imidazol-2-yl]-pyridine

1-(4-Chloro-phenyl)-2-pyridin-4-yl-ethane-1,2-dione2-oxime (261 mg, 1 mmol) was subjected in an analogous manner to the procedure described in Example 30, but using 3-pyridinecarboxaldehyde instead of bis-2,4-(trifluoromethyl)-benzaldehyde, to give the title compound (191 mg) as white solid. Mp 248-250° C.

### Example 32

### 4-[5-(4-Chloro-phenyl)-1-ethyl-4-pyridin-4-yl-1H-imidazol-2-yl]-quinoline

1-(4-Chloro-phenyl)-2-pyridin-4-yl-ethane-1,2-dione2-oxime (261 mg, 1 mmol) was subjected in an analogous manner to the procedure described in Example 30, but using 4-quinolinecarboxaldehyde instead of bis-2,4-(trifluoromethyl)-benzaldehyde, to give the title compound (140 mg) white solid. MS (ISP) 411.5 [(M+H)⁺]; mp 167-169° C.

### Example 33

### 2-[5-(4-Chloro-phenyl)-1-ethyl-4-pyridin-4-yl-1H-imidazol-2-yl]-1H-indole

1-(4-Chloro-phenyl)-2-pyridin-4-yl-ethane-1,2-dione2-oxime (261 mg, 1 mmol) was subjected in an analogous manner to the procedure described in Example 30, but using 1H-indole-2-carboxaldehyde instead of bis-2,4-(trifluoromethyl)-benzaldehyde, to give the title compound (23 mg) as light-yellow solid. MS (ISP) 399.3 [(M+H)⁺]; mp 279-280° C.

### Example 34

### 4-[2-tert-Butyl-5-(4-chloro-phenyl)-1-ethyl-1H-imidazol-4-yl]-pyridine

1-(4-Chloro-phenyl)-2-pyridin-4-yl-ethane-1,2-dione2-oxime (261 mg, 1 mmol) was subjected in an analogous manner to the procedure described in Example 30, but using 2,2-dimethyl-propanal instead of bis-2,4-(trifluoromethyl)-benzaldehyde, to give the title compound (4 mg) as white solid. MS (ISP) 340.3 [(M+H)⁺].

### Example 35

### 4-[5-(4-Chloro-phenyl)-1-ethyl-2-(1-ethyl-propyl)-1H-imidazol-4-yl]-pyridine

1-(4-Chloro-phenyl)-2-pyridin-4-yl-ethane-1,2-dione2-oxime (261 mg, 1 mmol) was subjected in an analogous manner to the procedure described in Example 30, but using 2-ethyl-butanal instead of bis-2,4-(trifluoromethyl)-benzaldehyde, to give the title compound (197 mg) as pale-yellow solid. Mp 239-242° C.

### Example 36

### 4-[5-(4-Chloro-phenyl)-2-(1-ethyl-propyl)-1-methyl-1H-imidazol-4-yl]-pyridine

1-(4-Chloro-phenyl)-2-pyridin-4-yl-ethane-1,2-dione2-oxime (261 mg, 1 mmol) was subjected in an analogous manner to the procedure described in Example 35, but using an 8 M solution of methylamine in ethanol (0.38 mL) instead of 30% aqueous ethylamine, to give the title compound (205 mg) as pale-yellow solid. MS (ISP) 340.3 [(M+H)⁺]; mp 100-102° C.

### Example 37

### 4-[5-(4-Chloro-phenyl)-1-ethyl-2-(1-propyl-butyl)-1H-imidazol-4-yl]-pyridine

1-(4-Chloro-phenyl)-2-pyridin-4-yl-ethane-1,2-dione2-oxime (261 mg, 1 mmol) was subjected in an analogous manner to the procedure described in Example 30, but using 2-propyl-pentanal instead of bis-2,4-(trifluoromethyl)-benzaldehyde, to give the title compound (83 mg) as white solid. MS (ISP) 382.4 [(M+H)⁺]; mp 152-155° C.

### Example 38

### 4-[5-(4-Chloro-phenyl)-1-ethyl-2-(1-isobutyl-3-methyl-butyl)-1H-imidazol-4-yl]-pyridine

1-(4-Chloro-phenyl)-2-pyridin-4-yl-ethane-1,2-dione2-oxime (261 mg, 1 mmol) was subjected in an analogous manner to the procedure described in Example 30, but using 2-isobutyl-4-methyl-pentanal instead of bis-2,4-(trifluoromethyl)-benzaldehyde, to give the title compound (24 mg) as off-white solid. MS (ISP) 410.5 [(M+H)⁺].

### Example 39

### 4-[2-Benzhydryl-5-(4-chloro-phenyl)-1-ethyl-1H-imidazol-4-yl]-pyridine

A mixture of 1-(4-chloro-phenyl)-2-pyridin-4-yl-ethane-1,2-dione2-oxime (291 mg, 1 mmol), diphenyl-acetaldehyde (0.22 mL, 1.2 mmol), 2 M ethylamine/tetrahydrofuran (1.5 mL, 3.0 mmol), and acetic acid (3 mL), was heated to 110° C. for 5 h. Zinc powder (0.2 g) was added to the mixture and heating was continued for 3 h. The reaction mixture was cooled and the pH was set to 6 by the addition of 25% aqueous ammonia (15 mL). The mixture was extracted with ethyl acetate (40 mL), and the organic layer was washed with brine, dried over sodium sulfate, and evaporated in vacuo. The residue was chromatographed on silica gel using dichloromethane/methanol (20:1, v/v) as eluent to give the title compound (61 mg) as white solid. MS (ISP) 450.3 [(M+H)⁺]; mp 199-200° C.

### Example 40

### 3-[5-(4-Chloro-phenyl)-1-ethyl-4-pyridin-4-yl-1H-imidazol-2-yl]-pentan-3-ol

To a solution of 4-[2-bromo-5-(4-chloro-phenyl)-1-ethyl-1H-imidazol-4-yl]-pyridin (181 mg, 0.5 mol) in tetrahydrofuran (2 mL) was added at −78° C. 1.6 M buthyllithium-hexane solution (0.31 mL, 0.5 mol). The brownish suspension formed was stirred for 45 min at −78° C., and then, diethyl ketone was added. Stiring was continued for 2 h at −78° C. and the mixture was then warmed up to 20° C. over 30 min. The mixture was extracted with ethyl acetate (40 mL), and the organic layer was washed with brine, dried over sodium sulfate, and evaporated in vacuo. The residue was chromatographed on silica gel using ethyl acetate/methanol (20:1, v/v) as eluent to give the title compound (50 mg) as off-white solid. MS (ISP) 370.3 [(M+H)⁺]. The starting material was prepared in the following way:

### 40-A: 4-[5-(4-chloro-phenyl)-1-ethyl-2-hydroxy-1H-imidazol-4-yl]-pyridine

A mixture of 1-(4-chloro-phenyl)-2-pyridin-4-yl-ethane-1,2-dione2-oxime (10.17 g, 0.039 mol), 36% aqueous formaldehyde (6.61 mL, 0.088 mol), and 70% aqueous ethylamine (4.42 mL, 0.55 mol) in acetic acid (150 mL) was heated to 110° C. for 45 h. The reaction mixture was cooled to 20° C. and the pH was set to 9 by the addition of 25% aqueous ammonia (40 mL). The mixture was extracted with ethyl acetate, and the organic layer was washed with brine, dried over sodium sulfate, and evaporated in vacuo. The crude product was triturated with dichloromethane to give the title compound (9.05 g) as white solid. MS (ISP) 300.1 [(M+H)⁺].

### 40-B: 4-[2-Bromo-5-(4-chloro-phenyl)-1-ethyl-1H-imidazol-4-yl]-pyridine

A mixture of 4-[5-(4-chloro-phenyl)-1-ethyl-2-hydroxy-1H-imidazol-4-yl]-pyridine (9.0 g, 0.03 mol) and phosphoryl bromide (34.4 g, 0.12 mol) was heated to 110° C. for 4 h. The reaction mixture was cooled to 20° C. and triturated with diethyl ether (0.2 L). The ether solution was decanted and discarded. The insoluble residue was dissolved in ethyl acetate (0.4 L) and the solution was washed with saturated sodium bicarbonate solution (0.25 L) for 15 min. The organic layer was washed with brine, dried over sodium sulfate, and evaporated in vacuo. The crude product was triturated with tert.-butyl methyl ether to give the title compound (10.65 g) as white solid. MS (ISP) 362.1 [(M+H)⁺].

### Example 41

### 3-[5-(4-Chloro-phenyl)-1-ethyl-4-pyridin-4-yl-1H-imidazol-2-yl]-2,4-dimethyl-pentan-3-ol

4-[2-bromo-5-(4-chloro-phenyl)-1-ethyl-1H-imidazol-4-yl]-pyridine (181 mg, 0.5 mol) was subjected in an analogous manner to the procedure described in Example 40, but replacing diethyl ketone by diisopropyl ketone, to give the title compound (34 mg) as off-white solid. MS (ISP) 398.4 [(M+H)⁺]; mp 221-223° C.

### Example 42

### 4-[5-(4-Chloro-phenyl)-1-ethyl-4-pyridin-4-yl-1H-imidazol-2-yl]-hepta-1,6-dien-4-ol

In an analogous manner, but replacing diethyl ketone by diallyl ketone, 4-[2-bromo-5-(4-chloro-phenyl)-1-ethyl-1H-imidazol-4-yl]-pyridine (181 mg, 0.5 mol) was subjected to the procedure described in Example 40, to give the title compound (34 mg) as off-white solid. MS (ISP) 394.3 [(M+H)⁺]; mp 159-160° C.

### Example 43

### [5-(4-Chloro-phenyl)-1-ethyl-4-pyridin-4-yl-1H-imidazol-2-yl]-dicyclopropyl-methanol

In an analogous manner, but replacing diethyl ketone by dicyclopropyl ketone, 4-[2-bromo-5-(4-chloro-phenyl)-1-ethyl-1H-imidazol-4-yl]-pyridine (181 mg, 0.5 mol) was subjected to the procedure described in Example 40, to give the title compound (145 mg) as off-white solid. MS (ISP) 394.1 [(M+H)⁺]; mp 185-188° C.

### Example 44

### [5-(4-Chloro-phenyl)-1-ethyl-4-pyridin-4-yl-1H-imidazol-2-yl]-diphenyl-methanol

In an analogous manner, but replacing diethyl ketone by benzophenone, 4-[2-bromo-5-(4-chloro-phenyl)-1-ethyl-1H-imidazol-4-yl]-pyridine (181 mg, 0.5 mol) was subjected to the procedure described in Example 40, to give the title compound (74 mg) as white solid. MS (ISP) 466.3 [(M+H)⁺]; mp 222-223° C.

### Example 45

### [5-(4-Chloro-phenyl)-1-ethyl-4-pyridin-4-yl-1H-imidazol-2-yl]-bis-(3-trifluoromethyl-phenyl)-methanol

In an analogous manner, but replacing diethyl ketone by 3,3′-bis(trifluoromethyl)benzophenone, 4-[2-bromo-5-(4-chloro-phenyl)-1-ethyl-1H-imidazol-4-yl]-pyridine (181 mg, 0.5 mol) was subjected to the procedure described in Example 40, to give the title compound (74 mg) as white solid. MS (ISP) 602.3 [(M+H)⁺]; mp 193-195° C.

### Example 46

### [5-(4-Chloro-phenyl)-1-ethyl-4-pyridin-4-yl-1H-imidazol-2-yl]-bis-(3-fluoro-phenyl)-methanol

In an analogous manner, but replacing diethyl ketone by 3,3′-difluoro-benzophenone, 4-[2-bromo-5-(4-chloro-phenyl)-1-ethyl-1H-imidazol-4-yl]-pyridine (181 mg, 0.5 mol) was subjected to the procedure described in Example 40, to give the title compound (100 mg) as white solid. MS (ISP) 502.1 [(M+H)⁺]; mp 225-227° C.

### Example 47

### [5-(4-Chloro-phenyl)-1-ethyl-4-pyridin-4-yl-1H-imidazol-2-yl]-dicyclohexyl-methanol

In an analogous manner, but replacing diethyl ketone by dicyclohexyl ketone, 4-[2-bromo-5-(4-chloro-phenyl)-1-ethyl-1H-imidazol-4-yl]-pyridine (181 mg, 0.5 mol) was subjected to the procedure described in Example 40, to give the title compound (145 mg) as off-white solid. MS (ISP) 478.4 [(M+H)⁺]; mp 221-224° C.

### Example 48

### 2-[5-(4-Chloro-phenyl)-1-ethyl-4-pyridin-4-yl-1H-imidazol-2-yl]-1,3-diphenyl-propan-2-ol

In an analogous manner, but replacing diethyl ketone by dibenzyl ketone, 4-[2-bromo-5-(4-chloro-phenyl)-1-ethyl-1H-imidazol-4-yl]-pyridine (181 mg, 0.5 mol) was subjected to the procedure described in Example 40, to give the title compound (20 mg) as white solid. MS (ISP) 494.5 [(M+H)⁺]; mp 116-117° C.

### Example 49

### 4-[5-(4-Chloro-phenyl)-2-(2,6-dichloro-phenyl)-1-propyl-1H-imidazol-4-yl]-pyridine

The product of Example 22 (66 mg) was hydrogenated at atmospheric pressure in ethyl acetate (5 mL) in the presence of 5% palladium-charcoal (20 mg) for 1 h. The catalyst was filtered off, the solution was evaporated in vacuo and the residue was crystallized from ethyl acetate/hexane to give the title compound (36 mg) as white solid. MS (ISP) 442.3 [(M+H)⁺]; mp 220-222° C.

### Example 50

### 4-[2-(2,6-Dichloro-phenyl)-1-propyl-5-(4-methyl-phenyl)-1H-imidazol-4-yl]-pyridine

The product of Example 19 (84 mg) was subjected in an analogous manner to the procedure described in Example 49 to give the title compound (64 mg) as white solid. MS (ISP) 422.3 [(M+H)⁺]; mp 200-201° C.

### Example 51

### 4-[1-Propyl-5-(4-methyl-phenyl)-2-(2,4,6-trimethyl-phenyl)-1H-imidazol-4-yl]-pyridine

The product of Example 23 (59 mg) was subjected in an analogous manner to the procedure described in Example 49 to give the title compound (53 mg) as white foam. MS (ISP) 396.5 [(M+H)⁺]; mp 134-136° C.

### Example 52

### 2-[[5-(4-Chloro-phenyl)-1-ethyl-4-pyridin-4-yl-1H-imidazol-2-yl]-bis-(3-trifluoromethyl-phenyl)-methoxy]-ethanol

A mixture of the product of Example 45 (301 mg), ethylene glycol (0.5 mL), and conc. sulfuric acid (0.5 mL) in toluene (5 mL) was heated for 1 h to 110° C. The cooled mixture was poured into sat. sodium carbonate solution (20 ml) and the product was extracted with ethyl acetate. The organic layer was washed with brine, dried over sodium sulfate, and evaporated in vacuo. The residue was chromatographed on silica gel using ethyl acetate/heptane (1:1, v/v) as eluent to give the title compound (51 mg) as off-white solid. MS (ISP) 646.3 [(M+H)⁺]; mp 222-223° C.

### Example 53

### 4-[2-[Bis-(3-fluoro-phenyl)-methyl]-5-(4-chloro-phenyl)-1-ethyl-1H-imidazol-4-yl]-pyridine

A solution of the product of Example 46 (30 mg, 0.06 mmol) and of triethylsilane (0.15 mL, 0.94 mmol) in trifluoroacetic acid was heated to 70° C. for 40 h. The mixture was evaporated in vacuo and the residue was dissolved in ethyl acetate. The solution was washed with sat. sodium bicarbonate solution and with brine, dried over sodium sulfate, and evaporated in vacuo. The residue was chromatographed on silica gel using ethyl acetate/hexane (1:1, v/v) as eluent to give the title compound (24 mg) as off-white solid. MS (ISP) 486.4 [(M+H)⁺]; mp 184-186° C.

### Example 54

### 2-Chloro-4-[5-(4-chloro-phenyl)-2-(2,6-dichloro-phenyl)-1-ethyl-1H-imidazol-4-yl]-pyridine

A mixture of 1-(4-chloro-phenyl)-2-(2-chloro-pyridin-4-yl-ethane-1,2-dione2-oxime (148 mg, 0.5 mmol), 2,6-dichloro-benzaldehyde (105 mg, 0.6 mmol), 70% aqueous ethylamine (0.2 mL, 2.5 mmol), and acetic acid (2 mL) was subjected in an analogous manner to the procedure described in Example 30 to give the title compound (133 mg) as white solid. MS (ISP) 462.0 [(M+H)⁺]; mp 187-189° C.

The starting material was prepared in the following way:

### 54-A: 1-(4-Chloro-phenyl)-2-(2-chloro-pyridin-4-yl-ethane-1,2-dione

To a solution of diisopropylamine (3.37 mL, 24.0 mol) in THF (70 mL) was added at −78° C. over 5 min 1.6 M n-butyl lithium/hexane solution (13.7 mL, 22.0 mol). After 15 min, a solution of 2-chloro-4-methyl-pyridine (1.80 mL, 20 mol) in THF (5 mL) was added at −78° C. and the red solution was stirred for 1 h at this temperature. A solution of methyl 4-chloro-benzoate (5.20 g, 30 mol) in THF (20 mL), pre-cooled to −30° C., was added over 25 min. The mixture was warmed up to 20° C. over 30 min and stirring was continued for 2 h, whereupon a yellow precipitate was formed. The mixture was poured into ice-water and the pH set to 7 by the addition of 3 N HCl. The product was extracted with ethyl and the organic layer was washed with brine, dried over sodium sulfate, and evaporated in vacuo. The residue was chromatographed on silica gel using ethyl acetate/hexane (1:1, v/v) as eluent to give the title compound (0.74 g) as white solid. MS (ISP) 266.3 [(M+H)⁺].

### 54-B: (E/Z) 1-(4-Chloro-phenyl)-2-(2-chloro-pyridin-4-yl-ethane-1,2-dione2-oxime

To a solution of 1-(4-chloro-phenyl)-2-(2-chloro-pyridin-4-yl)-ethane-1,2-dione (0.74 g, 3.0 mmol) in acetic acid (15 mL) was added dropwise at 5-8° C. a solution of sodium nitrite (0.23 g, 3.0 mmol) in water (3 mL). The mixture was stirred for 40 min at 20° C. whereupon a water (15 mL) was added and stirring was continued for 15 min. The precipitate was isolated by filtration and dried to give the title compound (0.62 g, mixture of E/Z-isomers) as white solid.

### Example 55

### 4-[5-(4-Chloro-phenyl)-2-(2,6-dichloro-phenyl)-1-ethyl-1H-imidazol-4-yl]-2-methoxy-pyridine

A mixture of the product of Example 54 (93 mg, 0.2 mol), potassium hydroxide (22 mg, 0.2 mol), and methanol (0.016 mL, 0.4 mol) in DMSO was stirred for 7.5 h at 50° C. The solution was partitioned between ethyl acetate and water. The organic layer was washed with brine, dried over sodium sulfate, and evaporated in vacuo. The residue was chromatographed on silica gel using ethyl acetate/hexane (1:1, v/v) as eluent to give the title compound (65 mg) as white solid. MS (ISP) 458.1 [(M+H)⁺]; mp 138-140° C.

### Example I

Tablets of the following composition are produced in a conventional manner:

| | mg/Tablet |
|---|---|
| Active ingredient | 100 |
| Powdered. lactose | 95 |
| White corn starch | 35 |
| Polyvinylpyrrolidone | 8 |
| Na carboxymethylstarch | 10 |
| Magnesium stearate | 2 |
| Tablet weight | 250 |

### Example II

Tablets of the following composition are produced in a conventional manner:

| | mg/Tablet |
|---|---|
| Active ingredient | 200 |
| Powdered. lactose | 100 |
| White corn starch | 64 |
| Polyvinylpyrrolidone | 12 |
| Na carboxymethylstarch | 20 |
| Magnesium stearate | 4 |
| Tablet weight | 400 |

### Example III

Capsules of the following composition are produced:

| | mg/Capsule |
|---|---|
| Active ingredient | 50 |
| Crystalline. lactose | 60 |
| Microcrystalline cellulose | 34 |
| Talc | 5 |
| Magnesium stearate | 1 |
| Capsule fill weight | 150 |

The active ingredient having a suitable particle size, the crystalline lactose and the microcrystalline cellulose are homogeneously mixed with one another, sieved and thereafter talc and magnesium stearate are admixed. The final mixture is filled into hard gelatine capsules of suitable size.

1. A compound of formula I: 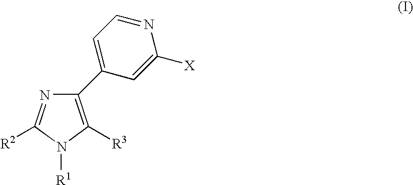
wherein
R¹ is a CHR^{a}R^{b} group in which:
either R^{a} is H and R^{b} is C₁₋₅-alkyl, C₂₋₅-alkenyl or C₂₋₅-alkynyl, each of which is optionally substituted by 1 to 5 substituents selected from the group consisting of F, Cl, Br, OH and OMe;
or R^{a} and R^{b} together with the carbon atom to which they are attached form a C₃₋₆-cycloalkyl ring; wherein R^{a} and R^{b} are each optionally substituted by 1 to 5 substituents selected from the group consisting of F, Cl, Br, OH and OMe;
R² is tert.-butyl;
or is —CR^{c}R^{d}R^{e}; wherein R^{c} and R^{d} are each independently branched or linear C₂₋₅-alkyl, C₂₋₅-alkenyl, C₃₋₆-cycloalkyl, phenyl, or benzyl, each of which is optionally substituted by 1 to 4 substituents selected from the group consisting of F, Cl, Br, OH, trifluoromethyl, C₁₋₄-alkyl and C₁₋₄-alkoxy; and R^{e} is H, OH, or a group OCH₂R^{f} wherein R^{f} is C₁₋₆-alkyl optionally substituted by OH;
or is aryl or heteroaryl, each of which is optionally substituted by 1 to 4 substituents selected from the group consisting of C₁₋₄-alkyl, trifluoromethyl, F, Cl, Br, OH, and C₁₋₄-alkoxy,
R³ is phenyl optionally substituted by 1 to 3 substituents selected from the group consisting of F, Cl, Br, trifluoromethyl and C₁₋₄-alkyl;
X is H, Cl, F or methoxy;
or a pharmaceutically acceptable salt thereof.

2. A compound of claim 1, wherein:
R¹ is —CH₂—C₁₋₅-alkyl optionally substituted by 1 to 5 substituents selected from the group consisting of F, Cl, Br, OH and OMe;
or a CHR^{a}R^{b} group wherein R^{a} and R^{b} together with the carbon atom to which they are attached form a C₃₋₆-cycloalkyl ring optionally substituted by 1 to 5 substituents selected from the group consisting of F, Cl, Br, OH and OMe;
R² is tert.-butyl;
or is —CR^{c}R^{d}R^{c}, wherein R^{c} and R^{d} are each independently branched or linear C₂₋₆-alkyl, C₂₋₆-alkenyl, C₃₋₆-cycloalkyl, phenyl, or benzyl, each of which is optionally substituted by 1 to 4 substituents selected from the group consisting of F, Cl, Br, trifluoromethyl, C₁₋₄-alkyl and C₁₋₄-alkoxy; and R^{e} is H, OH, or a group OCH₂R^{f} wherein R^{f} is C₁₋₆-alkyl optionally substituted by OH;
or is aryl or heteroaryl, each of which is optionally substituted by 1 to 4 substituents selected from the group consisting of C₁₋₄-alkyl, trifluoromethyl, F, Cl, Br, OH, and C₁₋₄-alkoxy;
R³ is phenyl optionally substituted by 1 to 3 substituents selected from the group consisting of F, Cl, Br, trifluoromethyl and C₁₋₄-alkyl;
X is H, Cl, F or methoxy;
or a pharmaceutically acceptable salt thereof.

3. A compound of claim 2, wherein R² is tert.-butyl.

4. A compound of claim 3, which is 4-[2-tert-Butyl-5-(4-chloro-phenyl)-1-ethyl-1H-imidazol-4-yl]-pyridine.

5. A compound of claim 2, wherein R² is —CR^{c}R^{d}R^{e}; wherein R^{c} and R^{d} are each independently branched or linear C₂₋₅-alkyl, C₂₋₅-alkenyl, C₃₋₆-cycloalkyl, phenyl, or benzyl, each of which is optionally substituted by 1 to 4 substituents selected from the group consisting of F, Cl, Br, trifluoromethyl, C₁₋₄-alkyl and C₁₋₄-alkoxy; and R^{e} is H, OH, or a group OCH₂R^{f} wherein R^{f} is C₁₋₆-alkyl optionally substituted by OH.

6. A compound of claim 5, selected from the group consisting of:
4-[5-(4-Chloro-phenyl)-1-ethyl-2-(1-ethyl-propyl)-1H-imidazol-4-yl]-pyridine;
4-[5-(4-Chloro-phenyl)-2-(1-ethyl-propyl)-1-methyl-1H-imidazol-4-yl]-pyridine;
4-[5-(4-Chloro-phenyl)-1-ethyl-2-(1-propyl-butyl)-1H-imidazol-4-yl]-pyridine;
4-[5-(4-Chloro-phenyl)-1-ethyl-2-(1-isobutyl-3-methyl-butyl)-1H-imidazol-4-yl]-pyridine;
4-[2-Benzhydryl-5-(4-chloro-phenyl)-1-ethyl-1H-imidazol-4-yl]-pyridine;
3-[5-(4-Chloro-phenyl)-1-ethyl-4-pyridin-4-yl-1H-imidazol-2-yl]-pentan-3-ol;
3-[5-(4-Chloro-phenyl)-1-ethyl-4-pyridin-4-yl-1H-imidazol-2-yl]-2,4-dimethyl-pentan-3-ol; and
4-[5-(4-Chloro-phenyl)-1-ethyl-4-pyridin-4-yl-1H-imidazol-2-yl]-hepta-1,6-dien-4-ol.

7. A compound of claim 5, selected from the group consisting of:
[5-(4-Chloro-phenyl)-1-ethyl-4-pyridin-4-yl-1H-imidazol-2-yl]-dicyclopropyl-methanol;
[5-(4-Chloro-phenyl)-1-ethyl-4-pyridin-4-yl-1H-imidazol-2-yl]-diphenyl-methanol;
[5-(4-Chloro-phenyl)-1-ethyl-4-pyridin-4-yl-1H-imidazol-2-yl]-bis-(3-trifluoromethyl-phenyl)-methanol;
[5-(4-Chloro-phenyl)-1-ethyl-4-pyridin-4-yl-1H-imidazol-2-yl]-bis-(3-fluoro-phenyl)-methanol;
[5-(4-Chloro-phenyl)-1-ethyl-4-pyridin-4-yl-1H-imidazol-2-yl]-dicyclohexyl-methanol;
2-[5-(4-Chloro-phenyl)-1-ethyl-4-pyridin-4-yl-1H-imidazol-2-yl]-1,3-diphenyl-propan-2-ol and
4-[2-[Bis-(3-fluoro-phenyl)-methyl]-5-(4-chloro-phenyl)-1-ethyl-1H-imidazol-4-yl]-pyridine.

8. A compound of claim 5, wherein R^{c} and R^{d} are each independently phenyl or benzyl, each of which is optionally substituted by 1 to 4 substituents selected from the group consisting of F, Cl, Br, trifluoromethyl, C₁₋₄-alkyl and C₁₋₄-alkoxy; and R^{e} is H, OH, or a group OCH₂R^{f} wherein R^{f} is C₁₋₆-alkyl optionally substituted by OH.

9. A compound of claim 2, wherein R² is optionally substituted aryl or heteroaryl, which when substituted is substituted by 1 to 4 substituents selected from the group consisting of C₁₋₄-alkyl, trifluoromethyl, F, Cl, Br and C₁₋₄-alkoxy.

10. A compound of claim 9, selected from the group consisting of
4-[2-(2,6-Dichloro-phenyl)-1-methyl-5-(4-methyl-phenyl)-1H-imidazol-4-yl]-pyridine;
4-[2-(2,6-Dichloro-phenyl)-1-propyl-5-(4-methyl-phenyl)-1H-imidazol-4-yl]-pyridine;
4-[1-Methyl-5-(4-methyl-phenyl)-2-(2,4,6-trimethyl-phenyl)-1H-imidazol-4-yl]-pyridine;
4-[5-(4-Chloro-phenyl)-2-(2,6-dichloro-phenyl)-1-methyl-1H-imidazol-4-yl]-pyridine;
4-[1-Ethyl-5-(4-methyl-phenyl)-2-(2,4,6-trimethyl-phenyl)-1H-imidazol-4-yl]-pyridine;
4-[5-(4-Chloro-phenyl)-2-(2,6-dichloro-phenyl)-1-ethyl-1H-imidazol-4-yl]-pyridine;
4-[1-Ethyl-5-(4-trifluoromethyl-phenyl)-2-(2,4,6-trimethyl-phenyl)-1H-imidazol-4-yl]-pyridine;
4-[5-(4-tert-Butyl-phenyl)-1-ethyl-2-(2,4,6-trimethyl-phenyl)-1H-imidazol-4-yl]-pyridine;
4-[5-(4-Chloro-phenyl)-1-ethyl-2-(2-methyl-phenyl)-1H-imidazol-4-yl]-pyridine; and
4-[5-(4-Chloro-phenyl)-1-ethyl-2-phenyl-1H-imidazol-4-yl]-pyridine.

11. A compound of claim 9, selected from the group consisting of
4-[1-Ethyl-5-(4-fluoro-phenyl)-2-(2,4,6-trimethyl-phenyl)-1H-imidazol-4-yl]-pyridine;
4-[2-(2,6-Dichloro-phenyl)-1-ethyl-5-(4-methyl-phenyl)-1H-imidazol-4-yl]-pyridine;
4-[5-(4-Chloro-phenyl)-1-ethyl-2-(2,4,6-trimethyl-phenyl)-1H-imidazol-4-yl]-pyridine;
4-[1-Ethyl-5-(4-trifluoromethyl-phenyl)-2-(2,4,6-trimethyl-phenyl)-1H-imidazol-4-yl]-pyridine;
4-[5-(4-Chloro-phenyl)-2-(2,6-dimethyl-phenyl)-1-ethyl-1H-imidazol-4-yl]-pyridine;
4-[5-(4-Chloro-phenyl)-2-(2-chloro-phenyl)-1-ethyl-1H-imidazol-4-yl]-pyridine;
4-[2-(2-Bromo-phenyl)-5-(4-chloro-phenyl)-1-ethyl-1H-imidazol-4-yl]-pyridine;
4-[2-(2-Chloro-6-methyl-phenyl)-5-(4-chloro-phenyl)-1-ethyl-1H-imidazol-4-yl]-pyridine;
4-[5-(4-Chloro-phenyl)-1-ethyl-2-(2,4,6-trimethoxy-phenyl)-1H-imidazol-4-yl]-pyridine; and
4-[1-Allyl-2-(2,6-dichloro-phenyl)-5-(4-methyl-phenyl)-1H-imidazol-4-yl]-pyridine.

12. A compound of claim 9, selected from the group consisting of
4-[1-Allyl-5-(4-trifluoromethyl-phenyl)-2-(2,4,6-trimethyl-phenyl)-1H-imidazol-4-yl]-pyridine;
4-[1-Allyl-5-(4-chloro-phenyl)-2-(2,4,6-trimethyl-phenyl)-1H-imidazol-4-yl]-pyridine;
4-[1-Allyl-5-(4-chloro-phenyl)-2-(2,6-dichloro-phenyl)-1H-imidazol-4-yl]-pyridine;
4-[1-Allyl-5-(4-methyl-phenyl)-2-(2,4,6-trimethyl-phenyl)-1H-imidazol-4-yl]-pyridine;
4-[5-(4-Chloro-phenyl)-1-cyclopentyl-2-(2,6-dichloro-phenyl)-1H-imidazol-4-yl]-pyridine;
4-[5-(4-Chloro-phenyl)-2-(2,6-dichloro-phenyl)-1-(2,2,2-trifluoro-ethyl)-1H-imidazol-4-yl]-pyridine;
2-[5-(4-Chloro-phenyl)-4-pyridin-4-yl-2-(2,4,6-trimethyl-phenyl)-imidazol-1-yl]-ethanol;
4-[5-(4-Chloro-phenyl)-1-cyclopropyl-2-(2,6-dichloro-phenyl)-1H-imidazol-4-yl]-pyridine;
4-[5-(4-Chloro-phenyl)-2-(2,6-dichloro-phenyl)-1-prop-2-ynyl-1H-imidazol-4-yl]-pyridine; and
3-[5-(4-Chloro-phenyl)-2-(2,6-dichloro-phenyl)-4-pyridin-4-yl-imidazol-1-yl]-propan-1-ol.

13. A compound of claim 9, selected from the group consisting of
4-[2-(2,4-Bis-trifluoromethyl-phenyl)-5-(4-chloro-phenyl)-1-ethyl-1H-imidazol-4-yl]-pyridine;
3-[5-(4-Chloro-phenyl)-1-ethyl-4-pyridin-4-yl-1H-imidazol-2-yl]-pyridine;
4-[5-(4-Chloro-phenyl)-1-ethyl-4-pyridin-4-yl-1H-imidazol-2-yl]-quinoline;
2-[5-(4-Chloro-phenyl)-1-ethyl-4-pyridin-4-yl-1H-imidazol-2-yl]-1H-indole;
4-[5-(4-Chloro-phenyl)-2-(2,6-dichloro-phenyl)-1-propyl-1H-imidazol-4-yl]-pyridine;
4-[2-(2,6-Dichloro-phenyl)-1-propyl-5-(4-methyl-phenyl)-1H-imidazol-4-yl]-pyridine;
4-[1-Propyl-5-(4-methyl-phenyl)-2-(2,4,6-trimethyl-phenyl)-1H-imidazol-4-yl]-pyridine
2-Chloro-4-[5-(4-chloro-phenyl)-2-(2,6-dichloro-phenyl)-1-ethyl-1H-imidazol-4-yl]-pyridine and
4-[5-(4-Chloro-phenyl)-2-(2,6-dichloro-phenyl)-1-ethyl-1H-imidazol-4-yl]-2-methoxy-pyridine.

14. A compound of claim 2, wherein R¹ is a CHR^{a}R^{b} group in which R^{a} is H and R^{b} is C₁₋₅-alkyl, C₂₋₅-alkenyl or C₂₋₅-alkynyl, each of which is optionally substituted by 1 to 5 substituents selected from the group consisting of F, Cl, Br, OH and OMe.

15. A compound of claim 14, wherein R^{b} is C₁₋₅-alkyl.

16. A compound of claim 2, wherein R¹ is a CHR^{a}R^{b} group in which R^{a} and R^{b} together with the carbon atom to which they are attached form a C₃₋₆-cycloalkyl ring; wherein R^{a} and R^{b} are each optionally substituted by 1 to 5 substituents selected from the group consisting of F, Cl, Br, OH and OMe.

17. A pharmaceutical composition comprising a compound of formula I 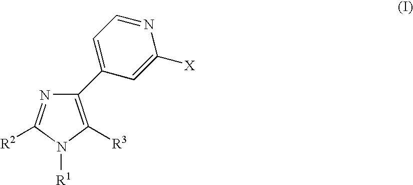
wherein
R¹ is a CHR^{a}R^{b} group in which:
either R^{a} is H and R^{b} is C₁₋₅-alkyl, C₂₋₅-alkenyl or C₂₋₅-alkynyl, each of which is optionally substituted by 1 to 5 substituents selected from the group consisting of F, Cl, Br, OH and OMe;
or R^{a} and R^{b} together with the carbon atom to which they are attached form a C₃₋₆-cycloalkyl ring; wherein R^{a} and R^{b} are each optionally substituted by 1 to 5 substituents selected from the group consisting of F, Cl, Br, OH and OMe;
R² is tert.-butyl;
or is —CR^{c}R^{d}R^{e}; wherein R^{c} and R^{d} are each independently branched or linear C₂₋₅-alkyl, C₂₋₅-alkenyl, C₃₋₆-cycloalkyl, phenyl, or benzyl, each of which is optionally substituted by 1 to 4 substituents selected from the group consisting of F, Cl, Br, OH, trifluoromethyl, C₁₋₄-alkyl and C₁₋₄-alkoxy; and R^{e} is H, OH, or a group OCH₂R^{f} wherein R^{f} is C₁₋₆-alkyl optionally substituted by OH;
or is aryl or heteroaryl, each of which is optionally substituted by 1 to 4 substituents selected from the group consisting of C₁₋₄-alkyl, trifluoromethyl, F, Cl, Br, OH, and C₁₋₄-alkoxy;
R³ is phenyl optionally substituted by 1 to 3 substituents selected from the group consisting of F, Cl, Br, trifluoromethyl and C₁₋₄-alkyl;
X is H, Cl, F or methoxy;
or a pharmaceutically acceptable salt thereof and a pharmaceutically acceptable carrier.
